(19) United States
(12) Patent Application Publication
(10) Pub. No.: US 2024/0398813 A1
(43) Pub. Date: Dec. 5, 2024

(54) PHARMACEUTICAL COMPOSITION FOR PREVENTING OR TREATING OVERACTIVE BLADDER

(71) Applicant: GWANGJU INSTITUTE OF SCIENCE AND TECHNOLOGY, Gwangju (KR)

(72) Inventors: CHUL SEUNG PARK, Gwangju (KR); JIN HEE AHN, Gwangju (KR); HEE JI JO, Gwangju (KR); EUN JUNG BAE, Gwangju (KR)

(21) Appl. No.: 18/697,452

(22) PCT Filed: May 10, 2022
(86) PCT No.: PCT/KR2022/006678

§ 371 Date: Mar. 29, 2024

(30) Foreign Application Priority Data
Dec. 13, 2021 (KR) 10-2021-0177953
May 2, 2022 (KR) 10-2022-0054412

(51) Int. Cl. A61K 31/519; A61P 13/06; C07D 513/04

(52) U.S. Cl. CPC A61K 31/519; A61P 13/06; C07D 513/04

(57) ABSTRACT

A quinazoline-based compound is represented by Formula 1 or Formula 2. The compound can effectively activate the BK_{Ca} channels, and thus may be used for preventing or treating the overactive bladder caused by inactivation or decrease in activity of the BK_{Ca} channels. The compound can be included in a pharmaceutical composition or health function food for improving urinary function.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/006678 filed on May 10, 2022, which claims priority to the benefit of Korean Patent Application Nos. 10-2021-0177953 filed on Dec. 13, 2021 and 10-2022-0054412 filed on May 2, 2022 in the Korean Intellectual Property Office, the entire contents of which are incorporated herein by reference.

### BACKGROUND

### 1. Technical Field

The present invention relates to a pharmaceutical composition for preventing or treating overactive bladder.

### 2. Background Art

BK_{Ca} channels are widely expressed in various types of excitable cells and nonexcitable cells, and involved in the regulation of several important physiological processes including neurotransmintter release (Raffaelli et al. 2006), smooth muscle contraction (Brenner et al. 2000; Herrera et al. 2000), and circadian behavioral rhythms (Meredith et al. 2006). Dysfunction of the BK_{Ca} channels is known to be the cause of several diseases such as epilepsy (Lorenz et al. 2007; Du et al. 2005), erectile dysfunction (Werner et al. 2005), and overactive bladder (OAB) (Meredith et al. 2004).

According to the definition of the International Continence Society, overactive bladder is a symptomatic disease based on the presence of urinary urgency (a symptom of feeling a strong and sudden need to urinate and not being able to hold urine when urinating), with or without urge incontinence (a symptom of not being able to hold urine and wetting one's pants a little when urinating) with no urinary tract infection or other obvious diseases, and accompanied by urinary frequency and nocturia. Overactive bladder syndrome mainly occurs in the elderly people, but recently, it has been known to occur frequently among people in their twenties and thirties who are under a lot of stress. The overactive bladder is not a serious disease, but if left untreated, it may cause lack of sleep, stress, depression and the like. Treatment of the overactive bladder includes methods such as behavioral therapy, drug therapy and the like. If the bladder does not respond to treatment, magnetic field therapy, bladder overdistention treatment, light alcohol injection, denervation surgery, augmentation cystoplasty, urinary diversion surgery, neuromodulation surgery, and the like may be performed.

Conventional therapeutic agents used to treat overactive bladder include antimuscarinic agents, beta-3 agonists, combination agents and the like. The antimuscarinic agent exerts a direct sedative effect on smooth muscles by competitively inhibiting the neurotransmitter acetylcholine from acting on muscarinic receptors. Thereby, the antimuscarinic agent is used to treat overactive bladder due to properties of increasing bladder capacity and residual urine volume and inhibiting bladder contraction, but may cause side effects such as dry mouth, drowsiness, constipation, dizziness, dry eyes and the like. Beta-3 agonists relax the bladder by stimulating beta-3 sympathetic receptors in the bladder, thereby used to treat overactive bladder, but may cause side effects such as urinary retention, urethral infection, hypertension and the like.

Accordingly, there is a need for research and development of new overactive bladder therapeutic agents to improve the side effects and limitations of these existing drugs.

### SUMMARY

An object of the present invention is to provide a pharmaceutical composition for preventing or treating overactive bladder, which includes a novel quinazoline-based compound.

Another object of the present invention is to provide a quinazoline-based compound, which is a novel BK_{Ca} channel activator.

1. A pharmaceutical composition for preventing or treating overactive bladder, including a compound represented by Formula 1 below, a stereoisomer or a pharmaceutically acceptable salt thereof:

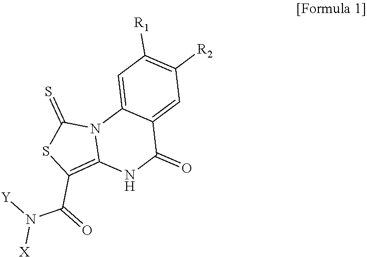

(wherein X is methyl, isopropyl, 2-chlorobenzyl, 4-methylbenzyl, 3-methoxybenzyl, 4-methoxybenzyl, cyclopentyl, (tetrahydrofuran-2-yl)methyl or substituted or unsubstituted phenyl, and Y is hydrogen or piperidine formed by linking with X, and
R₁ and R₂ are each independently hydrogen, methyl, halogen, methoxy or methoxycarbonyl, or R₁ and R₂ are 1,3-dioxolane formed by linking with each other).

2. The pharmaceutical composition according to the above 1, wherein the substituted phenyl is 2-methylphenyl, 3-methylphenyl, 4-methylphenyl, 2,4-dimethylphenyl, 5-chloro-2-methylphenyl, 2-ethylphenyl, 4-ethylphenyl, 2-methoxyphenyl, 3-methoxyphenyl, 2,5-dimethoxyphenyl, 2-ethoxyphenyl, 3-ethoxyphenyl, 4-(ethoxycarbonyl)phenyl, 3-fluorophenyl, 2,4-difluorophenyl, 4-bromo-2-fluorophenyl, 2-chlorophenyl, 3-chlorophenyl, 3-(trifluoromethyl)phenyl, 3-(trifluoromethoxy)phenyl or 3,5-bis(trifluoromethyl)phenyl.

3. The pharmaceutical composition according to the above 1, wherein R₁ is hydrogen and R₂ is Br.

4. The pharmaceutical composition according to the above 1, wherein the compound represented by Formula 1 above is any one selected from the group consisting of the following compounds:
7-bromo-5-oxo-1-thioxo-N-(m-tolyl)-4,5-dihydro-1H-thiazolo[3,4-a]quinazoline-3-carboxamide;
methyl 3-((3-fluorophenyl)carbamoyl)-5-oxo-1-thioxo-4,5-dihydro-1H-thiazolo[3,4-a]quinazoline-8-carboxylate;
ethyl 4-(8-chloro-5-oxo-1-thioxo-4,5-dihydro-1H-thiazolo[3,4-a]quinazoline-3-carboxamido)benzoate;
8-chloro-N-(2-ethylphenyl)-5-oxo-1-thioxo-4,5-dihydro-1H-thiazolo[3,4-a]quinazoline-3-carboxamide;
7-chloro-N-(2-methoxyphenyl)-5-oxo-1-thioxo-4,5-dihydro-1H-thiazolo[3,4-a]quinazoline-3-carboxamide;
7-bromo-N-isopropyl-5-oxo-1-thioxo-4,5-dihydro-1H-thiazolo[3,4-a]quinazoline-3-carboxamide;
N-(5-chloro-2-methylphenyl)-7-methyl-5-oxo-1-thioxo-4,5-dihydro-TH-thiazolo[3,4-a]quinazoline-3-carboxamide;
methyl 3-((2-chlorobenzyl)carbamoyl)-5-oxo-1-thioxo-4,5-dihydro-1H-thiazolo[3,4-a]quinazoline-8-carboxylate;
7-chloro-N-(2,4-dimethylphenyl)-5-oxo-1-thioxo-4,5-dihydro-1H-thiazolo[3,4-a]quinazoline-3-carboxamide;
7-bromo-N-(2,4-dimethylphenyl)-5-oxo-1-thioxo-4,5-dihydro-1H-thiazolo[3,4-a]quinazoline-3-carboxamide;
7-methyl-5-oxo-1-thioxo-N-(o-tolyl)-4,5-dihydro-1H-thiazolo[3,4-a]quinazoline-3-carboxamide;
N-(5-chloro-2-methylphenyl)-5-oxo-1-thioxo-4,5-dihydro-1H-[1,3]dioxolo[4,5-g]thiazolo[3,4-a]quinazoline-3-carboxamide;
methyl 3-((2,4-difluorophenyl)carbamoyl)-5-oxo-1-thioxo-4,5-dihydro-1H-thiazolo[3,4-a]quinazoline-8-carboxylate;
7-bromo-N-(4-methylbenzyl)-5-oxo-1-thioxo-4,5-dihydro-1H-thiazolo[3,4-a]quinazoline-3-carboxamide;
7-bromo-N-(2,5-dimethoxyphenyl)-5-oxo-1-thioxo-4,5-dihydro-1H-thiazolo[3,4-a]quinazoline-3-carboxamide;
7-bromo-5-oxo-N-((tetrahydrofuran-2-yl)methyl)-1-thioxo-4,5-dihydro-1H-[1,3]thiazolo[3,4-a]quinazoline-3-carboxamide;
N-(5-chloro-2-methylphenyl)-7,8-dimethoxy-5-oxo-1-thioxo-4,5-dihydro-1H-thiazolo[3,4-a]quinazoline-3-carboxamide;
N-(2,5-dimethoxyphenyl)-7-methyl-5-oxo-1-thioxo-4,5-dihydro-TH-thiazolo[3,4-a]quinazoline-3-carboxamide;
N-(2-ethylphenyl)-5-oxo-1-thioxo-4,5-dihydro-TH-[1,3]dioxolo[4,5-g]thiazolo[3,4-a]quinazoline-3-carboxamide;
7-bromo-N-(2-chlorobenzyl)-5-oxo-1-thioxo-4,5-dihydro-TH-thiazolo[3,4-a]quinazoline-3-carboxamide;
7-chloro-N-(2-chlorobenzyl)-5-oxo-1-thioxo-4,5-dihydro-TH-thiazolo[3,4-a]quinazoline-3-carboxamide;
7,8-dimethoxy-5-oxo-N-phenyl-1-thioxo-4,5-dihydro-1H-thiazolo[3,4-a]quinazoline-3-carboxamide;
8-chloro-N-cyclopentyl-5-oxo-1-thioxo-4,5-dihydro-1H-thiazolo[3,4-a]quinazoline-3-carboxamide;
N-(2-methoxyphenyl)-5-oxo-1-thioxo-4,5-dihydro-1H-[1,3]dioxolo[4,5-g]thiazolo[3,4-a]quinazoline-3-carboxamide;
8-chloro-N-methyl-5-oxo-1-thioxo-4,5-dihydro-1H-thiazolo[3,4-a]quinazoline-3-carboxamide;
N-(4-bromo-2-fluorophenyl)-8-chloro-5-oxo-1-thioxo-4,5-dihydro-1H-thiazolo[3,4-a]quinazoline-3-carboxamide;
7-chloro-5-oxo-N-((tetrahydrofuran-2-yl)methyl)-1-thioxo-4,5-dihydro-1H-thiazolo[3,4-a]quinazoline-3-carboxamide;
7-chloro-N-(2-ethoxyphenyl)-5-oxo-1-thioxo-4,5-dihydro-1H-thiazolo[3,4-a]quinazoline-3-carboxamide;
8-chloro-N-(3-methoxybenzyl)-5-oxo-1-thioxo-4,5-dihydro-1H-thiazolo[3,4-a]quinazoline-3-carboxamide;
7-bromo-3-(piperidine-1-carbonyl)-1-thioxo-1H-thiazolo[3,4-a]quinazolin-5(4H)-one;
N-(2,4-dimethylphenyl)-7-methyl-5-oxo-1-thioxo-4,5-dihydro-1H-thiazolo[3,4-a]quinazoline-3-carboxamide;
7-bromo-N-(4-ethylphenyl)-5-oxo-1-thioxo-4,5-dihydro-1H-thiazolo[3,4-a]quinazoline-3-carboxamide;
8-chloro-N-(4-methoxybenzyl)-5-oxo-1-thioxo-4,5-dihydro-1H-thiazolo[3,4-a]quinazoline-3-carboxamide;
8-chloro-N-(2,5-dimethoxyphenyl)-5-oxo-1-thioxo-4,5-dihydro-1H-thiazolo[3,4-a]quinazoline-3-carboxamide;
5-oxo-N-phenyl-1-thioxo-4,5-dihydro-1H-thiazolo[3,4-a]quinazoline-3-carboxamide;
8-methyl-5-oxo-N-phenyl-1-thioxo-4,5-dihydro-1H-thiazolo[3,4-a]quinazoline-3-carboxamide;
8-chloro-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-methyl-5-oxo-N-phenyl-1-thioxo-4,5-dihydro-1H-thiazolo[3,4-a]quinazoline-3-carboxamide;
7-chloro-5-oxo-N-phenyl-1-thioxo-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-5-oxo-1-thioxo-N-(p-tolyl)-4,5-dihydro-TH-thiazolo[3,4-a]quinazoline-3-carboxamide;
7-bromo-N-(3-methoxyphenyl)-5-oxo-1-thioxo-4,5-dihydro-TH-thiazolo[3,4-a]quinazoline-3-carboxamide;
7-bromo-N-(3-hydroxyphenyl)-5-oxo-1-thioxo-4,5-dihydro-TH-thiazolo[3,4-a]quinazoline-3-carboxamide;
7-bromo-N-(3-fluorophenyl)-5-oxo-1-thioxo-4,5-dihydro-TH-thiazolo[3,4-a]quinazoline-3-carboxamide;
7-bromo-N-(3-chlorophenyl)-5-oxo-1-thioxo-4,5-dihydro-TH-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

5. A health functional food for improving urinary function, including a compound represented by Formula 1 below or a stereoisomer thereof.

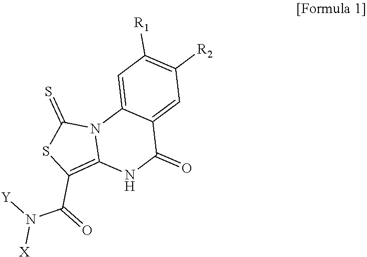

(wherein X is methyl, isopropyl, 2-chlorobenzyl, 4-methylbenzyl, 3-methoxybenzyl, 4-methoxybenzyl, cyclopentyl, (tetrahydrofuran-2-yl)methyl or substituted or unsubstituted phenyl, and Y is hydrogen or piperidine formed by linking with X, and
R₁ and R₂ are each independently hydrogen, methyl, halogen, methoxy or methoxycarbonyl, or R₁ and R₂ are 1,3-dioxolane formed by linking with each other).

6. The health functional food according to the above 5, wherein the substituted phenyl is 2-methylphenyl, 3-methylphenyl, 4-methylphenyl, 2,4-dimethylphenyl, 5-chloro-2-methylphenyl, 2-ethylphenyl, 4-ethylphenyl, 2-methoxyphenyl, 3-methoxyphenyl, 2,5-dimethoxyphenyl, 2-ethoxyphenyl, 3-ethoxyphenyl, 4-(ethoxycarbonyl)phenyl, 3-fluorophenyl, 2,4-difluorophenyl, 4-bromo-2-fluorophenyl, 2-chlorophenyl, 3-chlorophenyl, 3-(trifluoromethyl)phenyl, 3-(trifluoromethoxy)phenyl or 3,5-bis(trifluoromethyl)phenyl.

7. The health functional food according to the above 5, wherein R₁ is hydrogen and R₂ is Br.

8. The health functional food according to the above 5, wherein the compound represented by Formula 1 above is any one selected from the group consisting of the following compounds:
7-bromo-5-oxo-1-thioxo-N-(m-tolyl)-4,5-dihydro-1H-thiazolo[3,4-a]quinazoline-3-carboxamide;
methyl 3-((3-fluorophenyl)carbamoyl)-5-oxo-1-thioxo-4,5-dihydro-1H-thiazolo[3,4-a]quinazoline-8-carboxylate;
ethyl 4-(8-chloro-5-oxo-1-thioxo-4,5-dihydro-1H-thiazolo[3,4-a]quinazoline-3-carboxamido)benzoate;
8-chloro-N-(2-ethylphenyl)-5-oxo-1-thioxo-4,5-dihydro-1H-thiazolo[3,4-a]quinazoline-3-carboxamide;
7-chloro-N-(2-methoxyphenyl)-5-oxo-1-thioxo-4,5-dihydro-1H-thiazolo[3,4-a]quinazoline-3-carboxamide;
7-bromo-N-isopropyl-5-oxo-1-thioxo-4,5-dihydro-1H-thiazolo[3,4-a]quinazoline-3-carboxamide;
N-(5-chloro-2-methylphenyl)-7-methyl-5-oxo-1-thioxo-4,5-dihydro-1H-thiazolo[3,4-a]quinazoline-3-carboxamide;
methyl 3-((2-chlorobenzyl)carbamoyl)-5-oxo-1-thioxo-4,5-dihydro-1H-thiazolo[3,4-a]quinazoline-8-carboxylate;
7-chloro-N-(2,4-dimethylphenyl)-5-oxo-1-thioxo-4,5-dihydro-1H-thiazolo[3,4-a]quinazoline-3-carboxamide;
7-bromo-N-(2,4-dimethylphenyl)-5-oxo-1-thioxo-4,5-dihydro-1H-thiazolo[3,4-a]quinazoline-3-carboxamide;
7-methyl-5-oxo-1-thioxo-N-(o-tolyl)-4,5-dihydro-1H-thiazolo[3,4-a]quinazoline-3-carboxamide;
N-(5-chloro-2-methylphenyl)-5-oxo-1-thioxo-4,5-dihydro-1H-[1,3]dioxolo[4,5-g]thiazolo[3,4-a]quinazoline-3-carboxamide;
methyl 3-((2,4-difluorophenyl)carbamoyl)-5-oxo-1-thioxo-4,5-dihydro-1H-thiazolo[3,4-a]quinazoline-8-carboxylate;
7-bromo-N-(4-methylbenzyl)-5-oxo-1-thioxo-4,5-dihydro-1H-thiazolo[3,4-a]quinazoline-3-carboxamide;
7-bromo-N-(2,5-dimethoxyphenyl)-5-oxo-1-thioxo-4,5-dihydro-1H-thiazolo[3,4-a]quinazoline-3-carboxamide;
7-bromo-5-oxo-N-((tetrahydrofuran-2-yl)methyl)-1-thioxo-4,5-dihydro-1H-[1,3]thiazolo[3,4-a]quinazoline-3-carboxamide;
N-(5-chloro-2-methylphenyl)-7,8-dimethoxy-5-oxo-1-thioxo-4,5-dihydro-1H-thiazolo[3,4-a]quinazoline-3-carboxamide;
N-(2,5-dimethoxyphenyl)-7-methyl-5-oxo-1-thioxo-4,5-dihydro-1H-thiazolo[3,4-a]quinazoline-3-carboxamide;
N-(2-ethylphenyl)-5-oxo-1-thioxo-4,5-dihydro-1H-[1,3]dioxolo[4,5-g]thiazolo[3,4-a]quinazoline-3-carboxamide;
7-bromo-N-(2-chlorobenzyl)-5-oxo-1-thioxo-4,5-dihydro-1H-thiazolo[3,4-a]quinazoline-3-carboxamide;
7-chloro-N-(2-chlorobenzyl)-5-oxo-1-thioxo-4,5-dihydro-1H-thiazolo[3,4-a]quinazoline-3-carboxamide;
7,8-dimethoxy-5-oxo-N-phenyl-1-thioxo-4,5-dihydro-1H-thiazolo[3,4-a]quinazoline-3-carboxamide;
8-chloro-N-cyclopentyl-5-oxo-1-thioxo-4,5-dihydro-1H-thiazolo[3,4-a]quinazoline-3-carboxamide;
N-(2-methoxyphenyl)-5-oxo-1-thioxo-4,5-dihydro-1H-[1,3]dioxolo[4,5-g]thiazolo[3,4-a]quinazoline-3-carboxamide;
8-chloro-N-methyl-5-oxo-1-thioxo-4,5-dihydro-1H-thiazolo[3,4-a]quinazoline-3-carboxamide;
N-(4-bromo-2-fluorophenyl)-8-chloro-5-oxo-1-thioxo-4,5-dihydro-1H-thiazolo[3,4-a]quinazoline-3-carboxamide;
7-chloro-5-oxo-N-((tetrahydrofuran-2-yl)methyl)-1-thioxo-4,5-dihydro-1H-thiazolo[3,4-a]quinazoline-3-carboxamide;
7-chloro-N-(2-ethoxyphenyl)-5-oxo-1-thioxo-4,5-dihydro-1H-thiazolo[3,4-a]quinazoline-3-carboxamide;
8-chloro-N-(3-methoxybenzyl)-5-oxo-1-thioxo-4,5-dihydro-1H-thiazolo[3,4-a]quinazoline-3-carboxamide;
7-bromo-3-(piperidine-1-carbonyl)-1-thioxo-1H-thiazolo[3,4-a]quinazolin-5(4H)-one;
N-(2,4-dimethylphenyl)-7-methyl-5-oxo-1-thioxo-4,5-dihydro-1H-thiazolo[3,4-a]quinazoline-3-carboxamide;
7-bromo-N-(4-ethylphenyl)-5-oxo-1-thioxo-4,5-dihydro-1H-thiazolo[3,4-a]quinazoline-3-carboxamide;
8-chloro-N-(4-methoxybenzyl)-5-oxo-1-thioxo-4,5-dihydro-1H-thiazolo[3,4-a]quinazoline-3-carboxamide;
8-chloro-N-(2,5-dimethoxyphenyl)-5-oxo-1-thioxo-4,5-dihydro-1H-thiazolo[3,4-a]quinazoline-3-carboxamide;
5-oxo-N-phenyl-1-thioxo-4,5-dihydro-1H-thiazolo[3,4-a]quinazoline-3-carboxamide;
8-methyl-5-oxo-N-phenyl-1-thioxo-4,5-dihydro-1H-thiazolo[3,4-a]quinazoline-3-carboxamide;
8-chloro-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-methyl-5-oxo-N-phenyl-1-thioxo-4,5-dihydro-1H-thiazolo[3,4-a]quinazoline-3-carboxamide;
7-chloro-5-oxo-N-phenyl-1-thioxo-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-5-oxo-1-thioxo-N-(p-tolyl)-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

9. A compound represented by Formula 2 below, a stereoisomer or a pharmaceutically acceptable salt thereof:

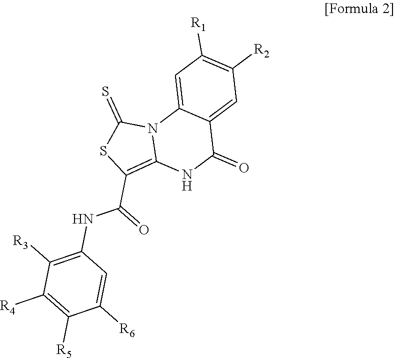

(wherein R₁ and R₂ are each independently hydrogen, methyl, or halogen, and R₃, R₄, R₅ and R₆ are each independently hydrogen, methyl, hydroxy, methoxy, halogen, trifluoromethyl or trifluoromethoxy).

10. The compound, or the stereoisomer or the pharmaceutically acceptable salt thereof according to the above 9, wherein R₁ is hydrogen and R₂ is Br.

11. The compound, or the stereoisomer or the pharmaceutically acceptable salt thereof according to the above 9, wherein the compound represented by Formula 2 above is any one compound selected from the group consisting of the following compounds:
8-methyl-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

The present invention provides a pharmaceutical composition for preventing or treating incontinence or overactive bladder, which includes a quinazoline-based compound.

The quinazoline-based compound of the present invention can activate BK_{Ca} channels, thus to induce relaxation of bladder smooth muscles and prevent excessive phasic contraction, and thereby may be used for prevention or treatment of the overactive bladder.

The quinazoline-based compound of the present invention has excellent activity, selectivity, and in vivo stability with low toxicity.

### BRIEF DESCRIPTION OF THE DRAWINGS

FIGS. 1A and 1B show representative structures and in vitro activity of quinazoline-based compounds of the present invention for activating BK_{Ca} channels. In FIG. 1A, respective compounds are described according to their activation effects on the BK_{Ca} channels in cell-based assay. The compounds were tested at 5 μM, wherein a positive control was LDD175 and a negative control was vehicle (DMSO). Changes (ARFU) in a relative fluorescence unit (RFU) were normalized by activities of LDD175. All compounds showed a significant p value<0.05 compared to a vehicle treatment group (n=3). In FIG. 1B, compounds were grouped into five subgroups (a-e). All compounds TTQC-1 to TTQC-34 include a common quinazoline ring.

FIG. 2A shows structures of TTQC-1, a quinazoline-based compound of the present invention, as well as rottlerin, NS11021, kurarinone, and LDD175, which are BK_{Ca} channel activators. FIG. 2B shows raw RFU signals after treatment of BK_{Ca} expressing cells with various BK_{Ca} activators and TTQC-1 at 3 μM. FIG. 2C shows changes (ARFU) in the RFU over 80 seconds after the channel stimulation was normalized by vehicle (DMSO)-treated cells. *p<0.05, **p<0.01, ***p<0.001 compared to the vehicle treatment group (n=4).

FIG. 3A shows raw RFU signals when BK_{Ca}-expressing cells were treated with 0.6-10 μM TTQC-1. Wherein the positive control was 5 μM NS11021, and the negative control was vehicle (DMSO). FIG. 3B shows changes (ARFU) in the RFU over 80 seconds after the channel stimulation was normalized by vehicle (DMSO) treatment. FIG. 3C shows signals after the raw RFUs were treated with 0.6-10 μM TTQC-1 and iberiotoxin (0.1 μM), a specific blocker of the BK_{Ca} channels. FIG. 3D shows that the ARFU during 80 seconds after the channel stimulation was normalized by the ARFU of vehicle treatment. **p<0.05, **p<0.01, **p<0.001 compared to the vehicle treatment group. ++p<0.01, ++p<0.001 compared to the iberiotoxin (0.1 μM) treatment group (n=4).

FIG. 4A shows relative BK_{Ca} channel current levels upon continuous perfusion and removal of TTQC-1 outside a cell membrane obtained from Xenopus oocytes. Wherein ion currents were generated every 1 second by pulses of +100 mV for 50 ms, with a sustaining potential of −100 mV. TTQC-1 was perfused at 10 μM (60-260 seconds) and washed with bath solution perfusion (260-510 seconds). The intracellular solution contains 3 μM Ca²⁺. Arrows indicate time points of representative current traces. FIG. 4B shows representative current traces at each time point (indicated by arrows a-d in FIG. 4A).

FIGS. 5A to 5D show that ion currents of the BK_{Ca} channels were induced by changes in step pulse voltages from −80 to +200 mV at an interval of 10 mV. Wherein the step pulse was maintained for 100 ms and the sustaining potential was −100 mV in the presence of 3 μM intracellular Ca²⁺. TTQC-1 was perfused outside the cell membrane at concentrations of 0, 0.1, 3, 10, and 30 μM. Specifically, FIG. 5A shows representative current traces from −80 to +180 mV upon perfusion of TTQC-1, wherein a gray line represents the ion current at +140 mV. FIG. 5B shows conductance-voltage (G-V) curves of the BK_{Ca} channels, which were normalized by the maximum conductance (G⁰ₘₐₓ) of vehicle (DMSO) treatment. The G-V curves were fitted by the Boltzmann equation: y={(Gₘₐₓ−Gₘᵢₙ)/(1+exp[(V_{1/2}−x)/k])}+Gₘᵢₙ, wherein k is RT/zF, and wherein R is a gas constant, T is a temperature, F is the Faraday's constant, and z is a gating charge. FIG. 5C shows the maximum conductance which was normalized by G° max. FIG. 5D shows a shifted half-maximum voltage (V_{1/2}) which was normalized by the voltage of vehicle treatment. ΔV_{1/2} was fitted by the Hill equation: y=ΔV_{1/2}, max x\hat{ }n/(EC₅₀\hat{ }n+x\hat{ }n), wherein n is the Hill coefficient and EC₅₀ values are each half value of the maximum effective concentration. *p<0.05, **p<0.01, ***p<0.001 compared to the vehicle treatment group (n=5 for vehicle and TTQC-1 0.1-10 μM; n=3 for NS11021 and TTQC-1 30 μM).

FIG. 6A shows initial outward currents of the BK_{Ca} channels at 150 mV upon perfusion of 10 μM TTQC-1 outside the cell membrane. FIG. 6B shows current traces which were fitted by an exponential equation: y(t)=A exp(−t/τ)+C channel activation time constant (i activation). FIG. 6C shows tail currents of the BK_{Ca} channels after a 150 mV pulse upon perfusion with 10 μM TTQC-1. FIG. 6D shows current traces which were fitted with the exponential equation for the time constant of channel deactivation (τ deactivation). *p<0.05, **p<0.01, ***p<0.001 compared to the vehicle treatment group (n=3-4).

FIGS. 7A to 7D show ion currents of BK_{Ca} channels co-expressed with 31 subunits. Wherein TTQC-1 was perfused at 10 μM outside the cell membrane, and the intracellular Ca²⁺ concentration was 3 μM. Specifically, FIG. 7A shows representative current traces of the BK_{Ca} channels co-expressed with the 31 subunits from −80 to +180 mV upon perfusion of TTQC-1, wherein a gray line represents the ion current at +140 mV. FIG. 7B shows conductance-voltage (G-V) curves of the BK_{Ca} channels, which were normalized by the maximum conductance (G⁰ₘₐₓ) of vehicle (DMSO) treatment. The G-V curve was fitted by the Boltzmann equation: y={(Gₘₐₓ−Gₘᵢₙ)/(1+exp[(V_{1/2}−x)/k])}+Gₘᵢₙ, wherein k is RT/zF, and wherein R is the gas constant, T is the temperature, F is the Faraday constant, and z is the gating charge. FIG. 7C shows time constants for channel activation (τ activation), which were fitted to initial outward currents using the exponential equation: y=A exp(−x/τ)+C. FIG. 7D shows time constants for channel deactivation (r deactivation), which were fitted to tail currents using the exponential equation. *p<0.05 compared to the vehicle treatment group (n=4-5).

FIGS. 8A to 8D show that activation effects of TTQC-1 depend on the co-expression of β4 subunits, and illustrates ion currents of BK_{Ca} channels co-expressed with the β4 subunits. Wherein TTQC-1 was perfused at 10 μM outside the cell membrane, and the intracellular Ca²⁺ concentration was 3 μM. Specifically, FIG. 8A shows representative current traces of the BK_{Ca} channels co-expressed with the β4 subunits from −80 to +180 mV upon perfusion of TTQC-1, wherein a gray line represents the ion current at +140 mV. FIG. 8B shows conductance-voltage (G-V) curves of the BK_{Ca} channels, which were normalized by the maximum conductance (G⁰ₘₐₓ) of vehicle (DMSO) treatment. The G-V curve was fitted by the Boltzmann equation. FIG. 8C shows time constants for channel activation (i activation), which were fitted to the initial outward current by the exponential equation: y=A exp(−x/τ)+C. FIG. 8D shows time constants for channel deactivation (i deactivation), which were fitted to the tail currents by the exponential equation. *p<0.05 compared to the vehicle treatment group (n=4-5).

FIG. 9A shows results of measuring the number of urinations in normal rats (WKY) and spontaneously hypertensive rats (SHR) for 3 hours after oral administration of TTQC-1 (10 or 50 mg/kg) or solifenacin succinate (5 mg/kg) as the positive control. The negative control was vehicles (DMSO, PEG400 and distilled water). FIG. 9B shows results of measuring the total urine volume for 3 hours of the normal rats and hypertensive rats orally administered TTQC-1 (10 or 50 mg/kg) or solifenacin succinate (5 mg/kg) as the positive control. The vehicles (DMSO, PEG400, and distilled water) were used as the negative control. ++p<0.01 compared to the WKY vehicle treatment group. **p<0.01 compared to the SHR vehicle treatment group (n=5).

FIG. 10A shows raw RFU signals upon treatment with 2 μM TTQC-1 in the presence or absence of paxilline and iberiotoxin. The negative control was vehicle and DMSO. FIG. 10B shows changes (ΔRFU) in the RFU over 80 seconds after channel stimulation normalized by the vehicle group. ++p<0.01 compared to vehicle group. ***p<0.001 compared with TTQC-1 (2 μM) group (n=4).

FIGS. 11A to 11C show results of the toxicity test of TTQC-1 through MTT analysis. Specifically, FIG. 11A shows cell survival rates when AD293 cells expressing hyperactive BK_{Ca} channels were treated with 0.1 to 25 μM TTQC-1 for 17 hours. FIG. 11B shows cell survival rates when HEK 293T cells were treated with 0.1 to 25 μM TTQC-1 for 17 hours. FIG. 11C shows cell survival rates when Hep G2 cells were treated with 0.1 to 25 μM TTQC-1 for 17 hours. *p<0.05, **p<0.01, *** p<0.001 compared to the vehicle (1% DMSO) treatment group (n=3-4).

FIG. 12 shows initial rates of channel activation for each compound. The initial rates of channel activation were obtained during the first 4 seconds after BK_{Ca} channel stimulation. Each compound was prepared at a concentration of 6 μM.

FIG. 13 shows apparent EC₅₀ values for each compound obtained at the initial rates of channel activation by fitting data to a dose-response curve, y=Amin+(Amax−Amin)/(1+10\hat{ }{(Log EC50−x)p}. NS11021 was used as the positive control.

FIGS. 14 and 15 show dose-dependent activation effects of Compound 208 on the BK_{Ca} channels. Specifically, FIG. 14 shows RFU after treatment in the concentration range of 0.2-6 μM. NS11021 (5 μM) was used as the positive control. FIG. 15 shows the initial rates of channel activation upon treatment with Compound 208 in each concentration range during the first 4 seconds. Student t-test was performed for statistical analysis. *p<0.05, **p<0.01, ***p<0.001 compared to the vehicle treatment group.

FIGS. 16A to 16D show activation effects of Compound 208 on the macroscopic currents of the BK_{Ca} channels. Wherein the currents were recorded in an outside-out configuration with an intracellular Ca²⁺ concentration of 3 μM. Ion currents were induced with 100 ms voltage step pulses from 80 mV to 200 mV with an increment of 10 mV. The sustaining voltage was 100 mV. Each trace corresponds to the voltage step. Specifically, FIG. 16A shows a representative current trace after treatment with 10 μM Compound 208. FIG. 16B shows conductance (G)-voltage (V) relationship curves after treatment with 10 μM Compound 208. The conductance (G) was obtained from the average outward current for 5 ms after saturation. The obtained values were normalized by the maximum conductance of the vehicle group by the Boltzmann function. G/Gₘₐₓ={(Gₘₐₓ−Gₘᵢₙ)/(1+exp[(V_{1/2}−V)/k])}+Gₘᵢₙ, wherein k is a constant. FIG. 16C shows a change in V_{1/2} (voltage at half activation) upon treatment with 10 μM Compound 208. FIG. 16D shows Gₘₐₓ/G^{o}ₘₐₓ upon treatment with 10 μM Compound 208. The maximum conductance (Gₘₐₓ) was increased at 10 μM Compound 208 compared to the vehicle treatment group (n=4). Student t-test was performed for statistical analysis. *p<0.05, **p<0.01, ***p<0.001 compared to the vehicle treatment group.

FIGS. 17A to 17C show effects of Compound 208 on BK_{Ca} channel gating. Specifically, FIG. 17A shows a representative current trace after treatment with 10 μM Compound 208 at 170 mV pulse stimulation. FIG. 17B shows the activation time constant (τ activation) according to the voltage. FIG. 17C shows the deactivation time constant (τ deactivation) according to the voltage. The tail current after peak was analyzed for τ deactivation after termination of the voltage pulse. The time constant (τ) was obtained by fitting all independent data sets using a single exponential function (y=A exp(−t/τ)+C). *p<0.05, **p<0.01, ***p<0.001 compared to the vehicle treatment group at the same voltage pulse.

FIG. 18 shows effects of Compound 208 on urinary behavior of spontaneously hypertensive rats. Urinary frequency was measured for 3 hours after oral administration in WKY and SHR. The vehicle consisted of DMSO:PEG400:distilled water (v/v, 5:40:55). *p<0.05, **p<0.01, ***p<0.001 compared to the vehicle group of SHR; +p<0.05, ++p<0.01, +++p<0.001 compared to the vehicle group of WKY.

### DETAILED DESCRIPTION

The present invention relates to a pharmaceutical composition for preventing or treating overactive bladder, which includes a compound represented by Formula 1 below, a stereoisomer or a pharmaceutically acceptable salt thereof.

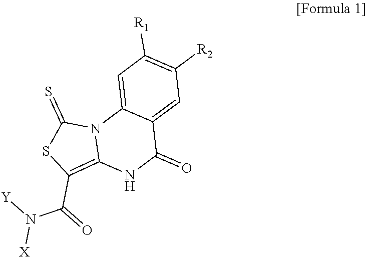

In Formula 1 above, X may be methyl, isopropyl, 2-chlorobenzyl, 4-methylbenzyl, 3-methoxybenzyl, 4-methoxybenzyl, cyclopentyl, (tetrahydrofuran-2-yl)methyl or substituted or unsubstituted phenyl, and Y may be hydrogen.

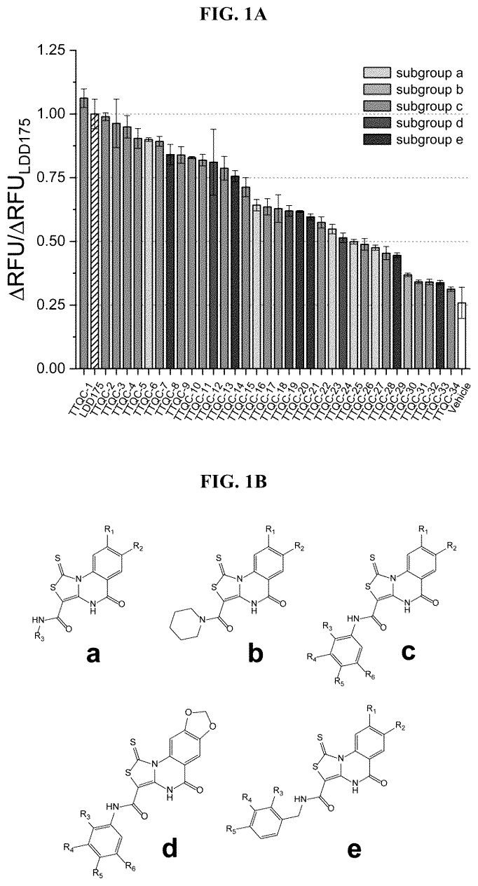

In Formula 1 above, X and Y may be linked with each other to form piperidine together with a nitrogen atom to which they are linked. Piperidine formed by linking X and Y with each other may have, for example, the structure as a compound b in FIG. 1B.

The substituted phenyl may be 2-methylphenyl, 3-methylphenyl, 4-methylphenyl, 2,4-dimethylphenyl, 5-chloro-2-methylphenyl, 2-ethylphenyl, 4-ethylphenyl, 2-methoxyphenyl, 3-methoxyphenyl, 2,5-dimethoxyphenyl, 2-ethoxyphenyl, 3-ethoxyphenyl, 4-(ethoxycarbonyl)phenyl, 3-fluorophenyl, 2,4-difluorophenyl, 4-bromo-2-fluorophenyl, 2-chlorophenyl, 3-chlorophenyl, 3-(trifluoromethyl)phenyl, 3-(trifluoromethoxy)phenyl or 3,5-bis(trifluoromethyl)phenyl.

In Formula 1 above, R₁ and R₂ may be each independently hydrogen, methyl, halogen, methoxy or methoxycarbonyl.

In Formula 1 above, R₁ and R₂ may be linked with each other to form 1,3-dioxolane, which may have, for example, the structure as a compound d in FIG. 1B.

In Formula 1 above, R₁ may be hydrogen and R₂ may be Br.

The compound represented by Formula 1 above may be any one selected from the group consisting of the following compounds:
7-bromo-5-oxo-1-thioxo-N-(m-tolyl)-4,5-dihydro-1H-thiazolo[3,4-a]quinazoline-3-carboxamide, TTQC-1;
methyl 3-((3-fluorophenyl)carbamoyl)-5-oxo-1-thioxo-4,5-dihydro-1H-thiazolo[3,4-a]quinazoline-8-carboxylate, TTQC-2;
ethyl 4-(8-chloro-5-oxo-1-thioxo-4,5-dihydro-1H-thiazolo[3,4-a]quinazoline-3-carboxamido)benzoate, TTQC-3
8-chloro-N-(2-ethylphenyl)-5-oxo-1-thioxo-4,5-dihydro-1H-thiazolo[3,4-a]quinazoline-3-carboxamide, TTQC-4;
7-chloro-N-(2-methoxyphenyl)-5-oxo-1-thioxo-4,5-dihydro-1H-thiazolo[3,4-a]quinazoline-3-carboxamide, TTQC-5;
7-bromo-N-isopropyl-5-oxo-1-thioxo-4,5-dihydro-1H-thiazolo[3,4-a]quinazoline-3-carboxamide, TTQC-6;
N-(5-chloro-2-methylphenyl)-7-methyl-5-oxo-1-thioxo-4,5-dihydro-1H-thiazolo[3,4-a]quinazoline-3-carboxamide, TTQC-7;
methyl 3-((2-chlorobenzyl)carbamoyl)-5-oxo-1-thioxo-4,5-dihydro-1H-thiazolo[3,4-a]quinazoline-8-carboxylate, TTQC-8;
7-chloro-N-(2,4-dimethylphenyl)-5-oxo-1-thioxo-4,5-dihydro-1H-thiazolo[3,4-a]quinazoline-3-carboxamide, TTQC-9;
7-bromo-N-(2,4-dimethylphenyl)-5-oxo-1-thioxo-4,5-dihydro-1H-thiazolo[3,4-a]quinazoline-3-carboxamide, TTQC-10;
7-methyl-5-oxo-1-thioxo-N-(o-tolyl)-4,5-dihydro-1H-thiazolo[3,4-a]quinazoline-3-carboxamide, TTQC-11;
N-(5-chloro-2-methylphenyl)-5-oxo-1-thioxo-4,5-dihydro-1H-[1,3]dioxolo[4,5-g]thiazolo[3,4-a]quinazoline-3-carboxamide, TTQC-12;
methyl 3-((2,4-difluorophenyl)carbamoyl)-5-oxo-1-thioxo-4,5-dihydro-1H-thiazolo[3,4-a]quinazoline-8-carboxylate, TTQC-13;
7-bromo-N-(4-methylbenzyl)-5-oxo-1-thioxo-4,5-dihydro-1H-thiazolo[3,4-a]quinazoline-3-carboxamide, TTQC-14;
7-bromo-N-(2,5-dimethoxyphenyl)-5-oxo-1-thioxo-4,5-dihydro-1H-thiazolo[3,4-a]quinazoline-3-carboxamide, TTQC-15;
7-bromo-5-oxo-N-((tetrahydrofuran-2-yl)methyl)-1-thioxo-4,5-dihydro-1H-[1,3]thiazolo[3,4-a]quinazoline-3-carboxamide, TTQC-16;
N-(5-chloro-2-methylphenyl)-7,8-dimethoxy-5-oxo-1-thioxo-4,5-dihydro-1H-thiazolo[3,4-a]quinazoline-3-carboxamide, TTQC-17;
N-(2,5-dimethoxyphenyl)-7-methyl-5-oxo-1-thioxo-4,5-dihydro-1H-thiazolo[3,4-a]quinazoline-3-carboxamide, TTQC-18;
N-(2-ethylphenyl)-5-oxo-1-thioxo-4,5-dihydro-1H-[1,3]dioxolo[4,5-g]thiazolo[3,4-a]quinazoline-3-carboxamide, TTQC-19;
7-bromo-N-(2-chlorobenzyl)-5-oxo-1-thioxo-4,5-dihydro-1H-thiazolo[3,4-a]quinazoline-3-carboxamide, TTQC-20;
7-chloro-N-(2-chlorobenzyl)-5-oxo-1-thioxo-4,5-dihydro-1H-thiazolo[3,4-a]quinazoline-3-carboxamide, TTQC-21;
7,8-dimethoxy-5-oxo-N-phenyl-1-thioxo-4,5-dihydro-1H-thiazolo[3,4-a]quinazoline-3-carboxamide, TTQC-22;
8-chloro-N-cyclopentyl-5-oxo-1-thioxo-4,5-dihydro-1H-thiazolo[3,4-a]quinazoline-3-carboxamide, TTQC-23;
N-(2-methoxyphenyl)-5-oxo-1-thioxo-4,5-dihydro-1H-[1,3]dioxolo[4,5-g]thiazolo[3,4-a]quinazoline-3-carboxamide, TTQC-24;
8-chloro-N-methyl-5-oxo-1-thioxo-4,5-dihydro-1H-thiazolo[3,4-a]quinazoline-3-carboxamide, TTQC-25;
N-(4-bromo-2-fluorophenyl)-8-chloro-5-oxo-1-thioxo-4,5-dihydro-1H-thiazolo[3,4-a]quinazoline-3-carboxamide, TTQC-26;
7-chloro-5-oxo-N-((tetrahydrofuran-2-yl)methyl)-1-thioxo-4,5-dihydro-1H-thiazolo[3,4-a]quinazoline-3-carboxamide, TTQC-27;
7-chloro-N-(2-ethoxyphenyl)-5-oxo-1-thioxo-4,5-dihydro-1H-thiazolo[3,4-a]quinazoline-3-carboxamide, TTQC-28;
8-chloro-N-(3-methoxybenzyl)-5-oxo-1-thioxo-4,5-dihydro-1H-thiazolo[3,4-a]quinazoline-3-carboxamide, TTQC-29;
7-bromo-3-(piperidine-1-carbonyl)-1-thioxo-1H-thiazolo[3,4-a]quinazolin-5(4H)-one, TTQC-30;
N-(2,4-dimethylphenyl)-7-methyl-5-oxo-1-thioxo-4,5-dihydro-1H-thiazolo[3,4-a]quinazoline-3-carboxamide, TTQC-31;
7-bromo-N-(4-ethylphenyl)-5-oxo-1-thioxo-4,5-dihydro-1H-thiazolo[3,4-a]quinazoline-3-carboxamide, TTQC-32;
8-chloro-N-(4-methoxybenzyl)-5-oxo-1-thioxo-4,5-dihydro-1H-thiazolo[3,4-a]quinazoline-3-carboxamide, TTQC-33;
8-chloro-N-(2,5-dimethoxyphenyl)-5-oxo-1-thioxo-4,5-dihydro-1H-thiazolo[3,4-a]quinazoline-3-carboxamide, TTQC-34;
5-oxo-N-phenyl-1-thioxo-4,5-dihydro-1H-thiazolo[3,4-a]quinazoline-3-carboxamide, Compound 101;
8-methyl-5-oxo-N-phenyl-1-thioxo-4,5-dihydro-1H-thiazolo[3,4-a]quinazoline-3-carboxamide, Compound 102;
8-chloro-5-oxo-N-phenyl-1-thioxo-4,5-dihydro-1H-thiazolo[3,4-a]quinazoline-3-carboxamide, Compound 103;
8-bromo-5-oxo-N-phenyl-1-thioxo-4,5-dihydro-1H-thiazolo[3,4-a]quinazoline-3-carboxamide, Compound 104;
7-methyl-5-oxo-N-phenyl-1-thioxo-4,5-dihydro-1H-thiazolo[3,4-a]quinazoline-3-carboxamide, Compound 105;
7-chloro-5-oxo-N-phenyl-1-thioxo-4,5-dihydro-1H-thiazolo[3,4-a]quinazoline-3-carboxamide, Compound 106;
7-bromo-5-oxo-N-phenyl-1-thioxo-4,5-dihydro-1H-thiazolo[3,4-a]quinazoline-3-carboxamide, Compound 107;
7-bromo-5-oxo-1-thioxo-N-(o-tolyl)-4,5-dihydro-1H-thiazolo[3,4-a]quinazoline-3-carboxamide, Compound 201;
7-bromo-5-oxo-1-thioxo-N-(p-tolyl)-4,5-dihydro-1H-thiazolo[3,4-a]quinazoline-3-carboxamide, Compound 203;
7-bromo-N-(3-methoxyphenyl)-5-oxo-1-thioxo-4,5-dihydro-1H-thiazolo[3,4-a]quinazoline-3-carboxamide, Compound 204;
7-bromo-N-(3-hydroxyphenyl)-5-oxo-1-thioxo-4,5-dihydro-1H-thiazolo[3,4-a]quinazoline-3-carboxamide, Compound 205;
7-bromo-N-(3-fluorophenyl)-5-oxo-1-thioxo-4,5-dihydro-1H-thiazolo[3,4-a]quinazoline-3-carboxamide, Compound 206;
7-bromo-N-(3-chlorophenyl)-5-oxo-1-thioxo-4,5-dihydro-1H-thiazolo[3,4-a]quinazoline-3-carboxamide, Compound 207;
7-bromo-5-oxo-1-thioxo-N-(3-(trifluoromethyl)phenyl)-4,5-dihydro-1H-thiazolo[3,4-a]quinazoline-3-carboxamide, Compound 208;
7-bromo-5-oxo-1-thioxo-N-(3-(trifluoromethoxy)phenyl)-4,5-dihydro-1H-thiazolo[3,4-a]quinazoline-3-carboxamide, Compound 209; and
N-(3,5-bis(trifluoromethyl)phenyl)-7-bromo-5-oxo-1-thioxo-4,5-dihydro-1H-thiazolo[3,4-a]quinazoline-3-carboxamide, Compound 210).

Specifically, the compound represented by Formula 1 above may be selected from the group consisting of the following compounds:
7-bromo-5-oxo-1-thioxo-N-(m-tolyl)-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

More specifically, the compound represented by Formula 1 above may be selected from the group consisting of the following compounds:
7-bromo-5-oxo-1-thioxo-N-(m-tolyl)-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;

The structures of the compounds TTQC-1 to TTQC-34 are as follows:

| Compound | Structure |
|---|---|
| TTQC- 1 | 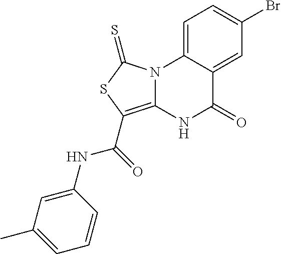 [SMILES: Cc1cccc(NC(=O)c2sc(=S)n3c2[nH]c(=O)c2cc(Br)ccc23)c1] |
| TTQC- 2 | 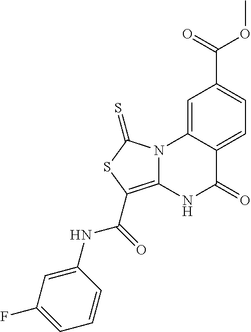 [SMILES: COC(=O)c1ccc2c(=O)[nH]c3c(C(=O)Nc4cccc(F)c4)sc(=S)n3c2c1] |
| TTQC- 3 | 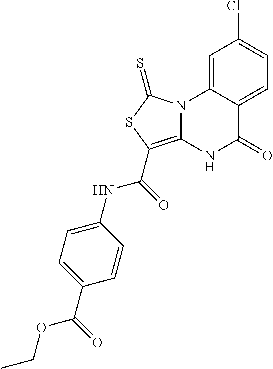 [SMILES: CCOC(=O)c1ccc(NC(=O)c2sc(=S)n3c2[nH]c(=O)c2ccc(Cl)cc23)cc1] |
| TTQC- 4 | 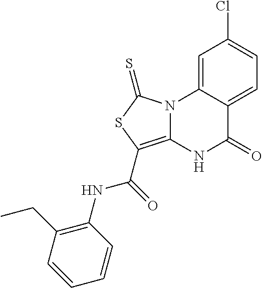 [SMILES: CCc1ccccc1NC(=O)c1sc(=S)n2c1[nH]c(=O)c1ccc(Cl)cc12] |
| TTOG- 5 | 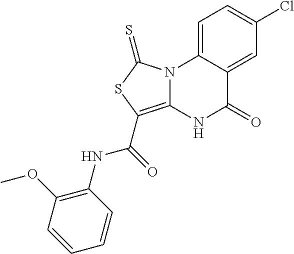 [SMILES: COc1ccccc1NC(=O)c1sc(=S)n2c1[nH]c(=O)c1cc(Cl)ccc12] |
| TTQC- 6 | 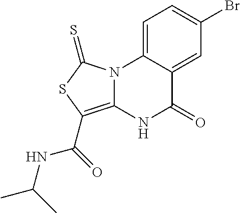 [SMILES: CC(C)NC(=O)c1sc(=S)n2c1[nH]c(=O)c1cc(Br)ccc12] |
| TTQC- 7 | 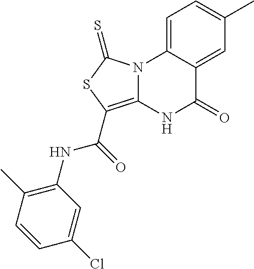 |
| ITQC- 8 | 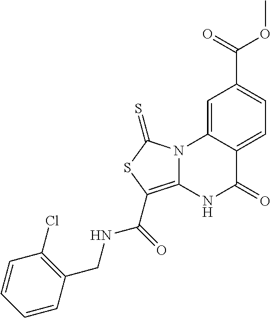 [SMILES: COC(=O)c1ccc2c(=O)[nH]c3c(C(=O)NCc4ccccc4Cl)sc(=S)n3c2c1] |
| TTQC- 9 | 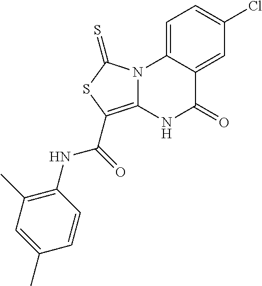 [SMILES: Cc1ccc(NC(=O)c2sc(=S)n3c2[nH]c(=O)c2cc(Cl)ccc23)c(C)c1] |
| TTQC- 10 | 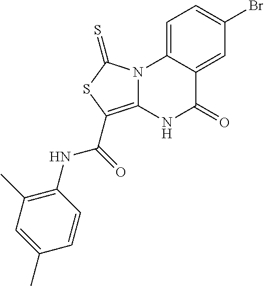 [SMILES: Cc1ccc(NC(=O)c2sc(=S)n3c2[nH]c(=O)c2cc(Br)ccc23)c(C)c1] |
| TTQC- 11 | 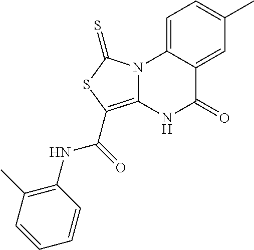 |
| TTQC- 12 | 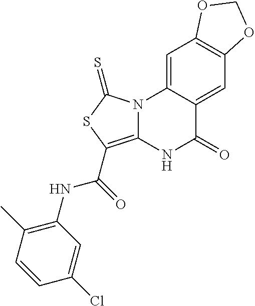 [SMILES: Cc1ccc(Cl)cc1NC(=O)c1sc(=S)n2c1[nH]c(=O)c1cc3c(cc12)OCO3] |
| TTQC- 13 | 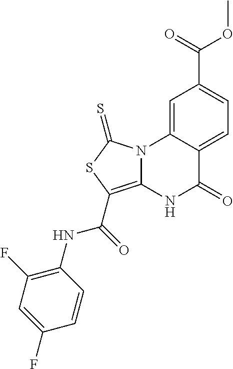 [SMILES: COC(=O)c1ccc2c(=O)[nH]c3c(C(=O)Nc4ccc(F)cc4F)sc(=S)n3c2c1] |
| TTQC- 14 | 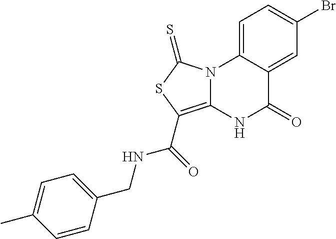 [SMILES: Cc1ccc(CNC(=O)c2sc(=S)n3c2[nH]c(=O)c2cc(Br)ccc23)cc1] |
| TTQC- 15 | 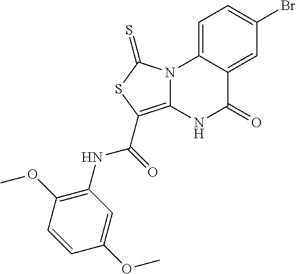 [SMILES: COc1ccc(OC)c(NC(=O)c2sc(=S)n3c2[nH]c(=O)c2cc(Br)ccc23)c1] |
| TTQC- 16 | 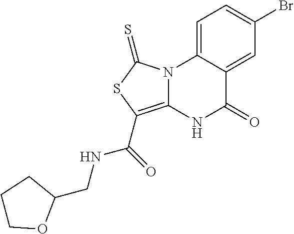 [SMILES: O=C(NCC1CCCO1)c1sc(=S)n2c1[nH]c(=O)c1cc(Br)ccc12] |
| TTQC- 17 | 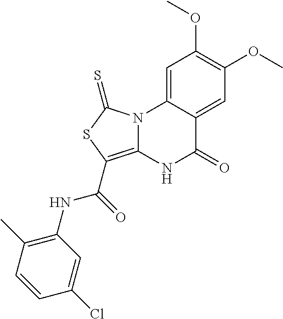 |
| TTQC- 18 | 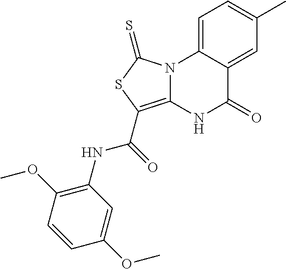 |
| TTQC- 19 | 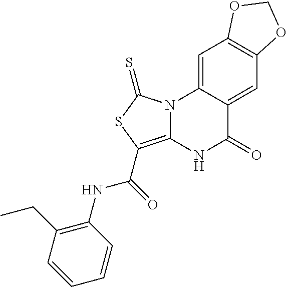 [SMILES: CCc1ccccc1NC(=O)c1sc(=S)n2c1[nH]c(=O)c1cc3c(cc12)OCO3] |
| TTQC- 20 | 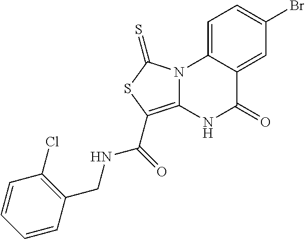 [SMILES: O=C(NCc1ccccc1Cl)c1sc(=S)n2c1[nH]c(=O)c1cc(Br)ccc12] |
| TTQC- 21 | 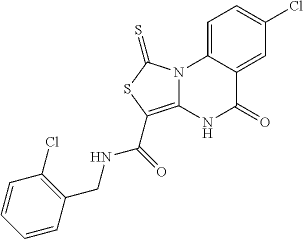 [SMILES: O=C(NCc1ccccc1Cl)c1sc(=S)n2c1[nH]c(=O)c1cc(Cl)ccc12] |
| TTQC- 22 | 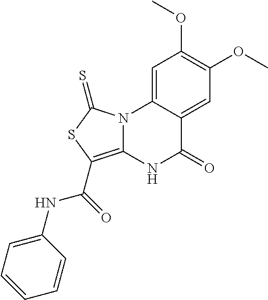 [SMILES: COc1cc2c(=O)[nH]c3c(C(=O)Nc4ccccc4)sc(=S)n3c2cc1OC] |
| TTQC- 23 | 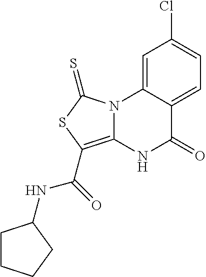 [SMILES: O=C(NC1CCCC1)c1sc(=S)n2c1[nH]c(=O)c1ccc(Cl)cc12] |
| TTQC- 24 | 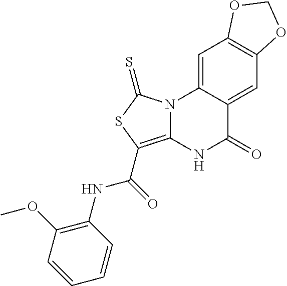 [SMILES: COc1ccccc1NC(=O)c1sc(=S)n2c1[nH]c(=O)c1cc3c(cc12)OCO3] |
| TTQC- 25 | 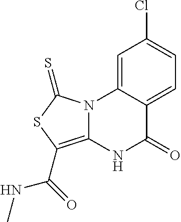 [SMILES: CNC(=O)c1sc(=S)n2c1[nH]c(=O)c1ccc(Cl)cc12] |
| TTQC- 26 | 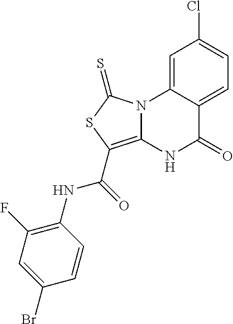 [SMILES: O=C(Nc1ccc(Br)cc1F)c1sc(=S)n2c1[nH]c(=O)c1ccc(Cl)cc12] |
| TTQC- 27 | 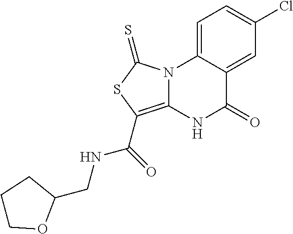 [SMILES: O=C(NCC1CCCO1)c1sc(=S)n2c1[nH]c(=O)c1cc(Cl)ccc12] |
| TTQC- 28 | 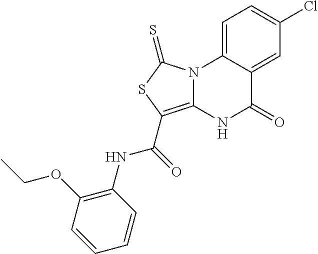 [SMILES: CCOc1ccccc1NC(=O)c1sc(=S)n2c1[nH]c(=O)c1cc(Cl)ccc12] |
| TTQC- 29 | 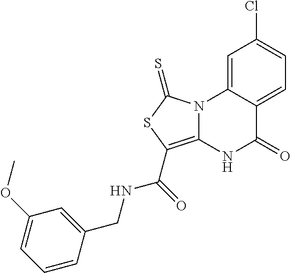 [SMILES: COc1cccc(CNC(=O)c2sc(=S)n3c2[nH]c(=O)c2ccc(Cl)cc23)c1] |
| TTQC- 30 | 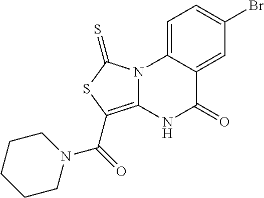 [SMILES: O=C(c1sc(=S)n2c1[nH]c(=O)c1cc(Br)ccc12)N1CCCCC1] |
| TTQC- 31 | 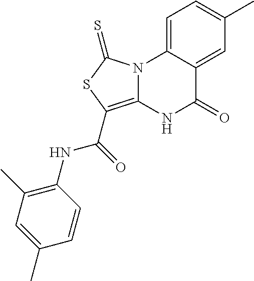 |
| TTQC- 32 | 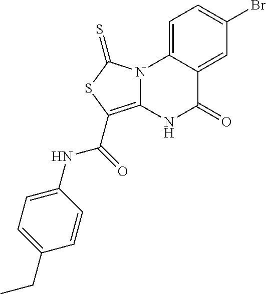 [SMILES: CCc1ccc(NC(=O)c2sc(=S)n3c2[nH]c(=O)c2cc(Br)ccc23)cc1] |
| TTQC- 33 | 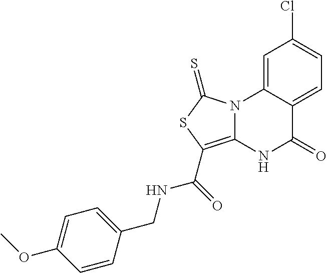 [SMILES: COc1ccc(CNC(=O)c2sc(=S)n3c2[nH]c(=O)c2ccc(Cl)cc23)cc1] |
| TTQC- 34 | 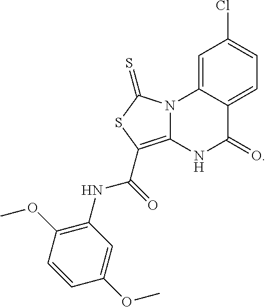 [SMILES: COc1ccc(OC)c(NC(=O)c2sc(=S)n3c2[nH]c(=O)c2ccc(Cl)cc23)c1] |

The structures of Compounds 101 to 107, 201 and 203 to 210 are as follows:

| Compound | Structure |
|---|---|
| 101 | 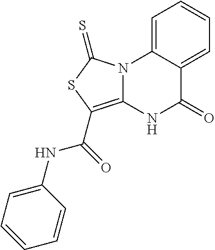 [SMILES: O=C(Nc1ccccc1)c1sc(=S)n2c1[nH]c(=O)c1ccccc12] |
| 102 | 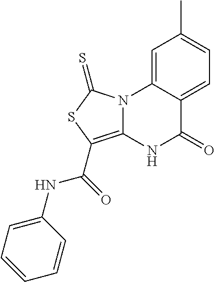 [SMILES: Cc1ccc2c(=O)[nH]c3c(C(=O)Nc4ccccc4)sc(=S)n3c2c1] |
| 103 | 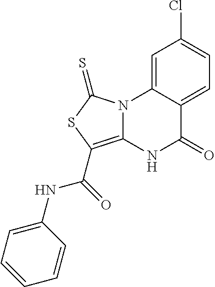 [SMILES: O=C(Nc1ccccc1)c1sc(=S)n2c1[nH]c(=O)c1ccc(Cl)cc12] |
| 104 | 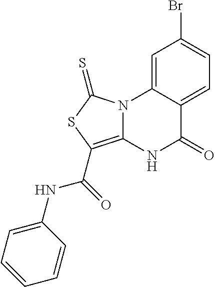 [SMILES: O=C(Nc1ccccc1)c1sc(=S)n2c1[nH]c(=O)c1ccc(Br)cc12] |
| 105 | 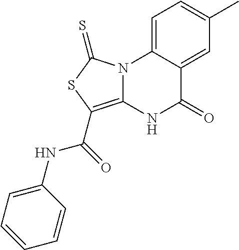 [SMILES: Cc1ccc2c(c1)c(=O)[nH]c1c(C(=O)Nc3ccccc3)sc(=S)n12] |
| 106 | 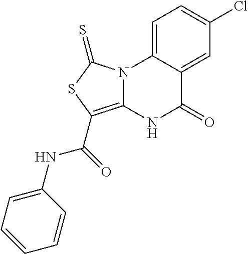 [SMILES: O=C(Nc1ccccc1)c1sc(=S)n2c1[nH]c(=O)c1cc(Cl)ccc12] |
| 107 | 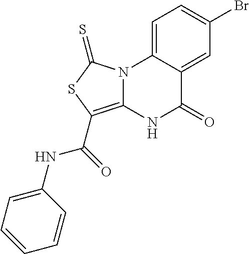 [SMILES: O=C(Nc1ccccc1)c1sc(=S)n2c1[nH]c(=O)c1cc(Br)ccc12] |
| 201 | 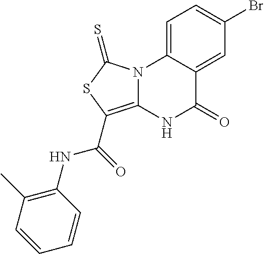 [SMILES: Cc1ccccc1NC(=O)c1sc(=S)n2c1[nH]c(=O)c1cc(Br)ccc12] |
| 203 | 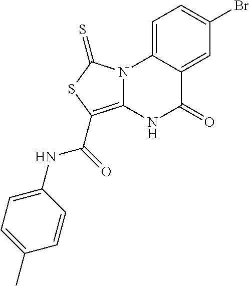 [SMILES: Cc1ccc(NC(=O)c2sc(=S)n3c2[nH]c(=O)c2cc(Br)ccc23)cc1] |
| 204 | 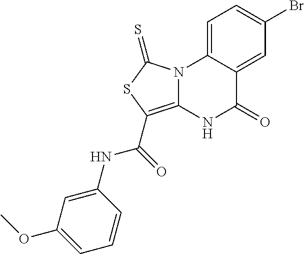 [SMILES: COc1cccc(NC(=O)c2sc(=S)n3c2[nH]c(=O)c2cc(Br)ccc23)c1] |
| 205 | 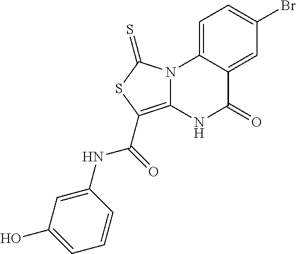 [SMILES: O=C(Nc1cccc(O)c1)c1sc(=S)n2c1[nH]c(=O)c1cc(Br)ccc12] |
| 206 | 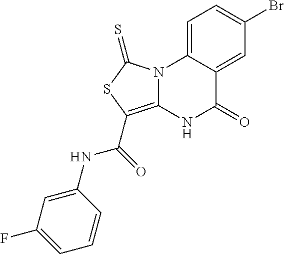 [SMILES: O=C(Nc1cccc(F)c1)c1sc(=S)n2c1[nH]c(=O)c1cc(Br)ccc12] |
| 207 | 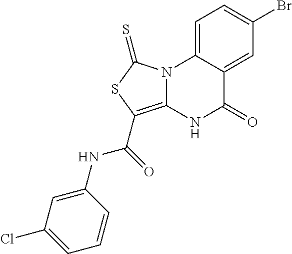 [SMILES: O=C(Nc1cccc(Cl)c1)c1sc(=S)n2c1[nH]c(=O)c1cc(Br)ccc12] |
| 208 | 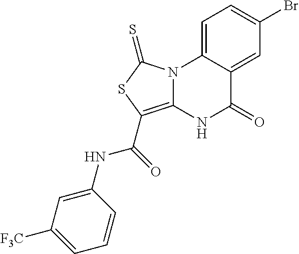 [SMILES: O=C(Nc1cccc(C(F)(F)F)c1)c1sc(=S)n2c1[nH]c(=O)c1cc(Br)ccc12] |
| 209 | 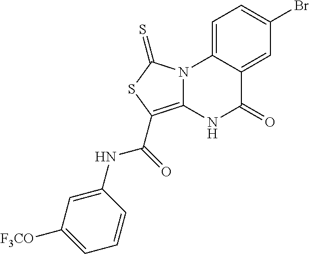 [SMILES: O=C(Nc1cccc(OC(F)(F)F)c1)c1sc(=S)n2c1[nH]c(=O)c1cc(Br)ccc12] |
| 210 | 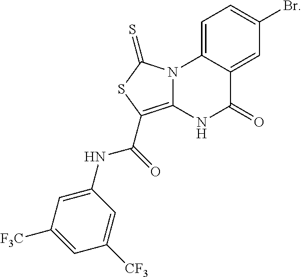 [SMILES: O=C(Nc1cc(C(F)(F)F)cc(C(F)(F)F)c1)c1sc(=S)n2c1[nH]c(=O)c1cc(Br)ccc12] |

The overactive bladder is a symptomatic disease based on the presence of urinary urgency (a symptom of feeling a strong and sudden need to urinate and not being able to hold urine when urinating), with or without urge incontinence (a symptom of not being able to hold urine and wetting one's pants a little when urinating) with no urinary tract infection or other obvious diseases, and accompanied by urinary frequency and nocturia.

The compound represented by Formula 1 above may induce relaxation of bladder smooth muscles by activating BK_{Ca} channels and prevent excessive phasic contraction of the bladder smooth muscles, and thereby may be used to prevent or treat overactive bladder.

The pharmaceutically acceptable carrier included in the composition of the present invention is generally used in producing a formulation, and may include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methylhydroxy benzoate, propylhydroxy benzoate, talc, magnesium stearate and mineral oil, etc., but it is not limited thereto. Other than the above components, the pharmaceutical composition of the present invention may further include lubricants, wetting agents, sweeteners, flavors, emulsifiers, suspending agents, preservatives and the like. Pharmaceutically acceptable carriers and formulations suitable in the art are described in detail in Remington's Pharmaceutical Sciences (19th ed., 1995). Suitable dosages of the pharmaceutical composition vary by factors such as a formulation method, administration manner, age, body weight, sex of a patient, severity of disease symptoms, food, administration time, administration route, excretion rate, response sensitization, or the like, and the dosage effective for desired treatment may be easily determined and prescribed by commonly skilled specialists in consideration of the above factors. Meanwhile, the dosage of the pharmaceutical composition of the present invention may be 0.01-2000 mg/kg (body weight) per day, but it is not limited thereto.

The pharmaceutical composition of the present invention may be administered orally or parenterally. For parenteral administration, intravenous injection, subcutaneous injection, intramuscular injection, intraperitoneal injection, transdermal administration, etc. may be used.

The pharmaceutical composition of the present invention may be formulated in a unit dose form using any pharmaceutically acceptable carrier and/or excipient, or may be formulated by introducing the same into a multi-dose container according to any method easily implemented by those skilled in the art, to which the present invention pertains. At this time, the formulation may have the form of a solution, suspension or emulsion in oil or aqueous medium, or the form of extract, powder, granules, tablets or capsules, and may further contain a dispersing agent or stabilizer.

The present invention relates to a health functional food for improving urinary function, which includes a compound represented by Formula 1 below or a stereoisomer thereof:

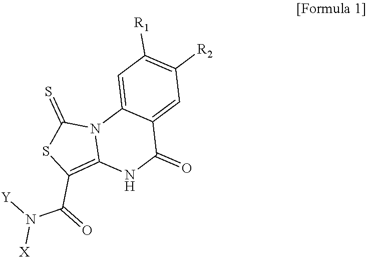

The compound represented by Formula 1 above is as described above.

The compound represented by Formula 1 above may induce relaxation of bladder smooth muscles by activating the BK_{Ca} channels and prevent excessive phasic contraction of the bladder smooth muscles, thereby improving urination function.

The health functional food of the present invention may include any conventional food additive. Herein, suitability of the health functional food as a food additive is judged on the basis of standards and criteria of corresponding items according to the General Regulations of the Food Additives and General Test Methods approved by the Food and Drug Administration, unless otherwise specified.

The items listed in the General Regulations of the Food Additives include, for example: chemical compounds such as ketones, glycine, calcium citrate, nicotinic acid and cinnamon acid; natural additives such as dark blue pigment, licorice extract, crystalline cellulose, high color pigment and guar gum; and mixed preparations such as sodium L-glutamate preparations, noodle-added alkaline chemicals, preservative preparations, and tar coloring preparations, and the like, but it is not limited thereto.

A health functional food in the form of tablets may be produced by mixing the extract with excipients, binders, disintegrants and other additives to prepare a mixture, granulating the mixture in any conventional manner, and then, compression molding the same along with addition of a lubricant or directly compression molding the mixture. In addition, the health functional food in the form of tablets may contain a flavor enhancer, or the like as necessary.

Among health functional foods in the form of capsules, a hard capsule formulation may be produced by filling a typical hard capsule with a mixture of the extract and additives such as excipients, and a soft capsule formulation may be produced by filling a capsule base such as gelatin with a mixture of the extract and additives such as excipients. The soft capsule formulation may further contain a plasticizer such as glycerin or sorbitol, a colorant, a preservative, and the like as necessary.

A health functional food in the form of pills may be produced by molding a mixture of the extract and excipients, binders, disintegrants, etc. according to any known method, and if necessary, may be enveloped with white sugar or other enveloping agents. Alternatively, the surface of the food may be coated with specific materials such as starch, talc and the like.

A health functional foods in the form of granules may be produced by granulating a mixture of the extract and excipients, binders, disintegrants, etc. according to a known method, and may contain a flavoring agent, a flavor enhancer, and the like as necessary.

Health functional foods may be beverages, meat, chocolate, foods, confectionery, pizza, ramen, other noodles, gums, candy, ice cream, alcoholic beverages, vitamin complexes and dietary supplements.

The health functional food may be orally applied for use of nutritional supplements, and the application forms thereof are not particularly limited. For example, for oral administration, daily intake is preferably 5000 mg or less, more preferably 2000 mg or less, and most preferably, 1000 mg or less. When formulated into capsules or tablets, one capsule or tablet may be administered along with water once a day.

The present invention relates to a compound represented by Formula 2 below, a stereoisomer or a pharmaceutically acceptable salt thereof:

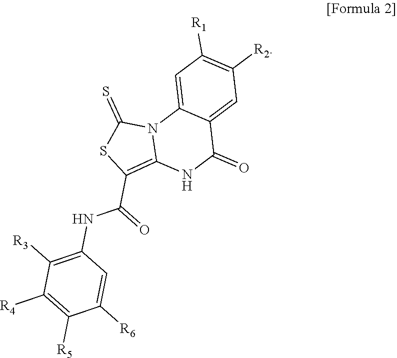

In Formula 2 above, R₁ and R₂ may be each independently hydrogen, methyl, or halogen, and more specifically, R₁ is hydrogen and R₂ is Br.

In Formula 2 above, R₃, R₄, R₅ and R₆ may be each independently hydrogen, methyl, hydroxy, methoxy, halogen, trifluoromethyl or trifluoromethoxy.

The compound represented by Formula 2 above may be any one selected from the group consisting of the following compounds:
5-oxo-N-phenyl-1-thioxo-4,5-dihydro-1H-thiazolo[3,4-a]quinazoline-3-carboxamide;
8-methyl-5-oxo-N-phenyl-1-thioxo-4,5-dihydro-1H-thiazolo[3,4-a]quinazoline-3-carboxamide;
8-chloro-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-methyl-5-oxo-N-phenyl-1-thioxo-4,5-dihydro-1H-thiazolo[3,4-a]quinazoline-3-carboxamide;
7-chloro-5-oxo-N-phenyl-1-thioxo-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-5-oxo-1-thioxo-N-(p-tolyl)-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-TH-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

More specifically, the compound represented by Formula 2 above may be any one selected from the group consisting of the following compounds:
8-methyl-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

Hereinafter, the present invention will be described in more detail through examples.

### PREPARATIVE EXAMPLE

### Preparative Example 1. Preparation of 1-thioxo-1H-thiazolo[3,4-a]quinazolin-5(4H)-one derivatives (Compounds 3 to 5)

### (1) Manufacturing Process

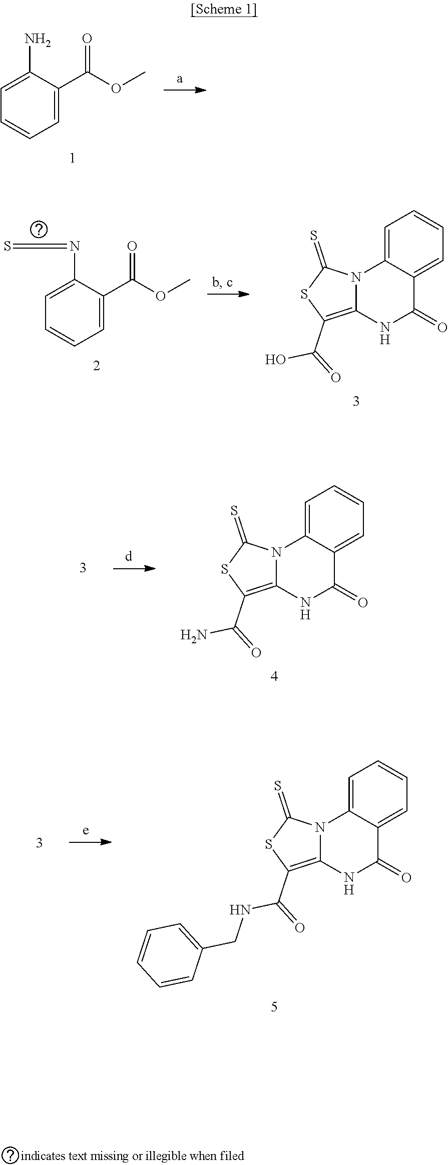 [SMILES: COC(=O)c1ccccc1N ; COC(=O)c1ccccc1N=C=S ; O=C(O)c1sc(=S)n2c1[nH]c(=O)c1ccccc12 ; NC(=O)c1sc(=S)n2c1[nH]c(=O)c1ccccc12 ; O=C(NCc1ccccc1)c1sc(=S)n2c1[nH]c(=O)c1ccccc12]

1-thioxo-1H-thiazolo[3,4-a]quinazolin-5(4H)-one derivatives (Compounds 3, 4, and 5) were synthesized according to Scheme 1. Commercially available methyl 2-aminobenzoate 1 was treated with thiophosgene to produce isothiocyanate intermediate 2, and then cyclized with methyl 2-cyanoacetate and sulfur to produce the corresponding ester compound. Thereafter, the ester compound was hydrolyzed to the corresponding carboxylic acid Compound 3 under basic conditions. Primary amide Compound 4 was formed by an amide coupling reaction of the carboxylic acid Compound 3 with HATU and ammonia solution. Benzyl amide Compound 5 was formed by EDCI coupling of carboxylic acid 3 with benzylamine formed.

Reagents and conditions. (a) thiophosgene, triethylamine, THF, 0° C. to 25° C., 1 h; (b) methyl 2-cyanoacetate, sulfur, triethylamine, DMF, 50° C., 1 h; (c) NaOH, THF, H2O, 25° C., 12 h; (d) 7M NH3 in MeOH, HATU, 1-Hydroxybenzotriazole, DIPEA, DMF, 25° C., 24 h; (e) benzylamine, EDCI, 1-Hydroxybenzotriazole, DIPEA, CH2Cl2, 25° C., 18 h.

### (2) Preparation of Compound 3 (5-oxo-1-thioxo-4,5-dihydro-1H-thiazolo[3,4-a]quinazoline-3-carboxylic acid)

Step 1) A solution prepared by mixing methyl 2-amino-5-bromobenzoate (1.27 g, 8.40 mmol) and triethylamine (2.34 mL, 16.80 mmol) in tetrahydrofuran (THF) was cooled to 0° C., followed by performing treatment by adding dropwise pure thiophosgene (0.68 mL, 8.82 mmol) thereto. The ice bath was removed and the reactant was stirred at ambient temperature for 1 hour. After completion of the reaction, the reaction mixture was evaporated. The reaction mixture was treated with NaHCO₃ aqueous solution and extracted with ethyl acetate. The mixed organic layer was dried over anhydrous sodium sulfate, then filtered and concentrated in vacuum to yield methyl 2-isothiocyanatobenzoate (Compound 2) (1.6 g, 99%) as a brown oil. Compound 2 was used in the next step without further purification.

Step 2) Methyl 2-cyanoacetate (820 mg, 8.28 mmol), sulfur (265 mg, 8.28 mmol) and trimethylamine (1.722 mL, 12.42 mmol) were mixed to a solution in which methyl 2-isothiocyanatobenzoate (Compound 2) (1.6 g, 8.28 mmol) was mixed and stirred in DMF. The reaction mixture was stirred at 50° C. for 1 hour. The reaction mixture was allowed to reach ambient temperature, diluted with ice water and acidified with acetic acid (3% v/v solution). The obtained solid was filtered and collected, then washed with ethanol to yield methyl 5-oxo-1-thioxo-4,5-dihydro-1H-thiazolo[3,4-a]quinazoline-3-carboxylate (1.53 g, 63%) as a yellow solid. An aqueous sodium hydroxide solution was added to a solution in which methyl 5-oxo-1-thioxo-4,5-dihydro-1H-thiazolo[3,4-a]quinazoline-3-carboxylate (1.42 g, 4.86 mmol) was mixed in tetrahydrofuran. The mixture was stirred at ambient temperature for 24 hours. The resulting mixture was evaporated to remove the solvent and acidified with iN hydrochloric acid to pH 2-3. Subsequently, the mixture was extracted twice with ethyl acetate, dried over anhydrous sodium sulfate, filtered and concentrated to yield 5-oxo-1-thioxo-4,5-dihydro-1H-thiazolo[3,4]-a]quinazoline-3-carboxylic acid (Compound 3) (570 mg, 42%) as a pale yellow solid.

1H NMR (400 MHz, DMSO-d₆) δ 10.82 (s, 1H), 10.57 (d, J=8.9 Hz, 1H), 8.23 (dd, J=7.8, 1.7 Hz, 1H), 7.95-7.91 (m, 1H), 7.70 (t, J=7.5 Hz, 1H)

### (3) Preparation of Compound 4 (5-oxo-1-thioxo-4,5-dihydro-1H-thiazolo[3,4-a]quinazoline-3-carboxamide)

Compound 3 (50 mg, 0.18 mmol) in DMF was mixed with HATU (171 mg, 0.45 mmol), HOBt (41 mg, 0.27 mmol) and N,N-diisopropylethylamine (0.11 mL, 0.63 mmol) and stirred at ambient temperature. After the mixture was stirred for 1 hour, 7M NH₃ (30 mg, 1.80 mmol) was added dropwise to MeOH and stirred for 24 hours. The reaction was monitored using thin layer chromatography. The resulting mixture was extracted with ethyl acetate and saturated NaHCO₃ solution. The mixed organic layer was dried over anhydrous sodium sulfate, filtered and evaporated. Then, the residue was purified by silica gel chromatography to yield 5-oxo-1-thioxo-4,5-dihydro-1H-thiazolo[3,4-a]quinazoline-3-carboxamide (Compound 4) (27 mg, 54%) as a yellow solid.

1H NMR (400 MHz, DMSO-d₆) δ 10.70 (dd, J=8.4, 0.8 Hz, 1H), 8.83 (d, J=3.8 Hz, 1H), 8.13 (dd, J=7.6, 1.9 Hz, 1H), 7.67-7.62 (m, 1H), 7.52 (td, J=7.4, 1.0 Hz, 1H), 7.22 (d, J=3.8 Hz, 1H)

### (4) Preparation of Compound 5 (N-benzyl-5-oxo-1-thioxo-4,5-dihydro-1H-thiazolo[3,4-a]quinazoline-3-carboxamide)

Benzylamine (23 mg, 0.22 mmol), EDCI (86 mg, 0.45 mmol), HOBt (41 mg, 0.27 mmol) and N,N-diisopropylethylamine 0.11 mL (0.63 mmol) were added to a solution in which Compound 3 (50 mg, 0.18 mmol) was mixed in dichloromethane and stirred at ambient temperature for 18 hours. The reaction mixture was diluted with water and extracted with dichloromethane. The mixed organic layer was dried over anhydrous sodium sulfate and evaporated. The residue was purified by silica gel column chromatography to yield N-benzyl-5-oxo-1-thioxo-4,5-dihydro-1H-thiazolo[3,4-a]quinazoline-3-carboxamide (Compound 5) (10 mg, 15%) as a yellow solid.

1H NMR (400 MHz, chloroform-d) δ 11.57 (s, 1H), 10.60 (d, J=8.2 Hz, 1H), 8.34 (dd, J=7.8, 1.7 Hz, 1H), 7.79-7.74 (m, 1H), 7.59-7.55 (m, 1H), 7.38-7.28 (m, 5H), 5.46 (s, 1H), 4.57 (d, J=5.8 Hz, 2H)

### Preparative Example 2. Preparation of 5-oxo-N-phenyl-1-thioxo-4,5-dihydro-1H-thiazolo[3,4-a]quinazoline-3-carboxamide derivatives (Compound TTQC-1, Compounds 101 to 107, 201 and 203 to 210)

### (1) Manufacturing Process

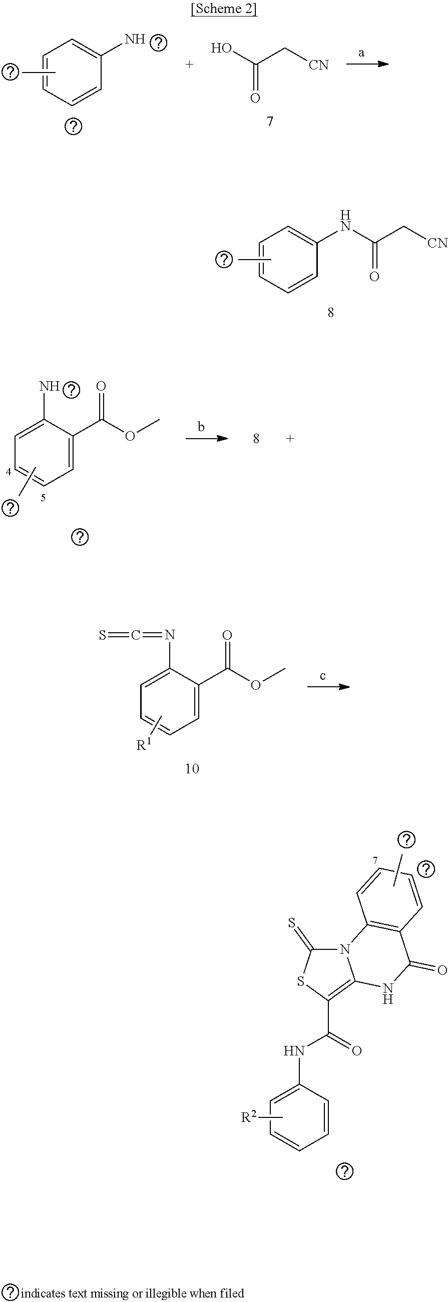

5-oxo-N-phenyl-1-thioxo-4,5-dihydro-1H-thiazolo[3,4-a]quinazoline-3-carboxamide derivative was synthesized according to Scheme 2. A cyano-acetamide intermediate (Compound 8) was produced by binding commercially available cyanoacetic acid (Compound 7) to aniline derivatives (Compounds 6a to 6k). Various amide derivatives (101 to 107 and 201 to 210) were obtained in two steps from a methyl 2-aminobenzoate derivative according to the procedures shown in Scheme 2. Compounds 9a to 9g as primary amines were converted to an isothiocyanate intermediate (Compound 10). Thereafter, ring-closed compounds (Compound TTQC-1, Compounds 101 to 107, 201 and 203 to 210) were obtained through cyclization of the intermediate (Compound 10) using cyanoacetamide (Compound 8) and sulfur.

Reagents and conditions: (a) EDCI, 1-Hydroxybenzotriazole, DIPEA, CH2Cl2, 25° C., 18 h; (b) thiophosgene, triethylamine, THF, 0° C. to 25° C., 1 h; (c) sulfur, triethylamine, DMF, 50° C., 1 h.

### (2) Preparation of Compound 8

A mixture, in which aniline (Compounds 6a-6k) (1.0 equivalent), 2-cyanoacetic acid (Compound 7), EDCI (2.5 equivalents), HOBt (1.5 equivalents) and N, N-diisopropylethylamine (3.5 equivalents) were mixed in dichloromethane, was stirred at ambient temperature for 18 hours. The reaction was monitored using thin layer chromatography. The resulting mixture was extracted with dichloromethane and saturated NaHCO₃ solution. The mixed organic layer was dried over anhydrous Na₂SO₄, filtered and evaporated. The unpurified substance was purified by silica gel chromatography to yield Compound 8.

### (3) Preparation of Compound TTQC-1, Compounds 101 to 107, 201 and 203 to 210

Step 1) A mixed solution prepared by mixing aniline (Compounds 9a-9g) and triethylamine (2.0 equivalent) in tetrahydrofuran was cooled to 0° C., followed by performing treatment by adding dropwise pure thiophosgene (1.05 equivalents) thereto. The ice bath was removed and the reactant was stirred at ambient temperature for 1 hour. The reaction was monitored using thin layer chromatography. The resulting mixture was diluted with water and saturated aqueous sodium bicarbonate. The resulting mixture was extracted with ethyl acetate. The mixed organic layer was dried with sodium sulfate, filtered, and concentrated under reduced pressure to yield isothiocyanatobenzene (Compound 10), which was used in the next step without further purification.

Step 2) A mixture, in which Compound 10 and the corresponding cyanoacetamide (Compound 8) (1.0 equivalent), sulfur (1.0 equivalent) and trimethylamine (1.5 equivalent) were mixed in DMF, was heated to 50° C. and stirred for 1 hour. The reaction mixture was allowed to reach ambient temperature, diluted with ice water and acidified with acetic acid (3% v/v solution). The obtained solid was collected by filtration and washed with ethanol to yield the compound.

### (4) NMR Data of Prepared Compounds

### 1) Compound TTQC-1(7-bromo-5-oxo-1-thioxo-N-(m-tolyl)-4,5-dihydro-1H-thiazolo[3,4-a]quinazoline-3-carboxamide)

yellow solid (54 mg, 16%). 1H NMR (400 MHz, DMSO-d₆) δ 10.56 (d, J=9.2 Hz, 1H), 10.18 (s, 1H), 8.23 (d, J=2.4 Hz, 1H), 8.09 (dd, J=9.2, 2.4 Hz, 1H), 7.47 (s, 1H), 7.44 (d, J=8.2 Hz, 1H), 7.23 (t, J=7.8 Hz, 1H), 6.94 (d, J=7.3 Hz, 1H), 2.30 (s, 3H)

### 2) Compound 101 (5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 11.48 (s, 1H), 10.67 (d, J=8.4 Hz, 1H), 8.20 (dd, J=8.0, 1.9 Hz, 1H), 7.76-7.73 (m, 1H), 7.60 (q, J=6.9 Hz, 3H), 7.34 (t, J=7.6 Hz, 2H), 7.06 (t, J=7.2 Hz, 1H)

### 3) Compound 102 (8-methyl-5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 10.49 (s, 1H), 10.12 (s, 1H), 8.10 (d, J=7.6 Hz, 1H), 7.64 (d, J=7.6 Hz, 2H), 7.50 (d, J=7.6 Hz, 1H), 7.36 (t, J=8.0 Hz, 2H), 7.14 (t, J=7.6 Hz, 1H), 2.49 (s, 3H)

### 4) Compound 103 (8-chloro-5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 10.82 (s, 1H), 10.21 (s, 1H), 8.20 (d, J=8.2 Hz, 1H), 7.77 (dd, J=8.5, 1.8 Hz, 1H), 7.65 (d, J=7.6 Hz, 2H), 7.37 (t, J=7.8 Hz, 2H), 7.15 (t, J=7.5 Hz, 1H)

### 5) Compound 104 (8-bromo-5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 10.96 (d, J=1.9 Hz, 1H), 10.24 (s, 1H), 8.12 (d, J=8.4 Hz, 1H), 7.91 (dd, J=8.4, 1.9 Hz, 1H), 7.65 (d, J=7.6 Hz, 2H), 7.37 (t, J=7.8 Hz, 2H), 7.15 (t, J=7.4 Hz, 1H)

### 6) Compound 105 (7-methyl-5-oxo-N-phenyl-1-thioxo-4,5-dihydro-1H-thiazolo[3,4-a]quinazoline-3-carboxamide)

yellow solid. 1H NMR (400 MHz, methanol-d₄) δ 10.54 (d, J=8.5 Hz, 1H), 8.00 (d, J=1.8 Hz, 1H), 7.75 (dd, J=8.7, 1.1 Hz, 2H), 7.42 (dd, J=8.5, 1.8 Hz, 1H), 7.34-7.30 (m, 2H), 7.06 (t, J=7.5 Hz, 1H), 2.42 (s, 3H)

### 7) Compound 106 (7-chloro-5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 10.83 (d, J=1.9 Hz, 1H), 10.25 (s, 1H), 8.21 (d, J=8.4 Hz, 1H), 7.78 (dd, J=8.4, 1.9 Hz, 1H), 7.65 (d, J=7.6 Hz, 2H), 7.37 (t, J=7.8 Hz, 2H), 7.15 (t, J=7.2 Hz, 1H)

### 8) Compound 107 (7-bromo-5-oxo-N-phenyl-1-thioxo-4,5-dihydro-1H-thiazolo[3,4-a]quinazoline-3-carboxamide)

yellow solid. 1H NMR (400 MHz, DMSO-d₆) δ 10.59 (d, J=9.5 Hz, 1H), 10.39 (s, 1H), 8.24 (d, J=2.1 Hz, 1H), 8.09 (dd, J=9.2, 2.1 Hz, 1H), 7.64 (d, J=8.5 Hz, 2H), 7.37 (t, J=7.8 Hz, 2H), 7.13 (t, J=7.3 Hz, 1H)

### 9) Compound 201 (7-bromo-5-oxo-1-thioxo-N-(o-tolyl)-4,5-dihydro-1H-thiazolo[3,4-a]quinazoline-3-carboxamide)

yellow solid (78 mg, 24%). 1H NMR (400 MHz, methanol-d₄) δ 11.47 (s, 1H), 10.71 (d, J=9.2 Hz, 1H), 8.32 (d, J=2.4 Hz, 1H), 7.85-7.84 (m, 1H), 7.73 (dd, J=9.3, 2.6 Hz, 1H), 7.22 (d, J=7.3 Hz, 1H), 7.16 (t, J=7.6 Hz, 1H), 7.03 (t, J=7.5 Hz, 1H), 2.47 (s, 3H)

### 10) Compound 203 (7-bromo-5-oxo-1-thioxo-N-(p-tolyl)-4,5-dihydro-1H-thiazolo[3,4-a]quinazoline-3-carboxamide)

yellow solid (209 mg, 64%). 1H NMR (400 MHz, DMSO-d₆) δ 10.59 (d, J=9.5 Hz, 1H), 10.28 (s, 1H), 10.14-10.46 (1H), 8.25 (d, J=2.3 Hz, 1H), 8.10 (d, J=9.2 Hz, 1H), 7.52 (d, J=8.4 Hz, 2H), 7.17 (d, J=8.4 Hz, 2H), 2.28 (s, 3H)

### 11) Compound 204 (7-bromo-N-(3-methoxyphenyl)-5-oxo-1-thioxo-4,5-dihydro-1H-thiazolo[3,4-a]quinazoline-3-carboxamide)

yellow solid (206 mg, 43%). 1H NMR (400 MHz, DMSO-d₆) δ 10.57 (d, J=9.2 Hz, 1H), 10.25 (s, 1H), 8.24 (d, J=2.4 Hz, 1H), 8.10 (dd, J=9.3, 2.6 Hz, 1H), 7.32 (s, 1H), 7.28-7.22 (m, 2H), 6.70 (td, J=4.7, 2.3 Hz, 1H), 3.75 (s, 3H)

### 12) Compound 205 (7-bromo-N-(3-hydroxyphenyl)-5-oxo-1-thioxo-4,5-dihydro-1H-thiazolo[3,4-a]quinazoline-3-carboxamide)

yellow solid (60 mg, 36%). 1H NMR (400 MHz, DMSO-d₆) δ 10.59 (d, J=9.2 Hz, 1H), 10.18 (s, 1H), 9.51 (s, 1H), 8.25 (d, J=2.4 Hz, 1H), 8.11 (dd, J=9.2, 2.4 Hz, 1H), 7.20 (s, 1H), 7.14 (t, J=7.9 Hz, 1H), 7.06 (d, J=8.2 Hz, 1H), 6.55-6.53 (m, 1H)

### 13) Compound 206 (7-bromo-N-(3-fluorophenyl)-5-oxo-1-thioxo-4,5-dihydro-1H-thiazolo[3,4-a]quinazoline-3-carboxamide)

yellow solid (112 mg, 37%). 1H NMR (400 MHz, DMSO-d₆) δ 10.57 (d, J=9.2 Hz, 1H), 10.47 (s, 1H), 8.25 (d, J=2.4 Hz, 1H), 8.11 (dd, J=9.3, 2.6 Hz, 1H), 7.63 (dt, J=11.6, 2.1 Hz, 1H), 7.46-7.37 (m, 2H), 6.99-6.94 (m, 1H)

### 14) Compound 207 (7-bromo-N-(3-chlorophenyl)-5-oxo-1-thioxo-4,5-dihydro-1H-thiazolo[3,4-a]quinazoline-3-carboxamide)

yellow solid (103 mg, 23%). 1H NMR (400 MHz, DMSO-d₆) δ 10.57 (d, J=9.2 Hz, 1H), 10.46 (s, 1H), 8.25 (s, 1H), 8.10 (dd, J=9.3, 2.6 Hz, 1H), 7.83 (s, 1H), 7.59 (d, J=7.6 Hz, 1H), 7.39 (t, J=8.1 Hz, 1H), 7.19 (d, J=7.9 Hz, 1H)

### 15) Compound 208 (7-bromo-5-oxo-1-thioxo-N-(3-(trifluoromethyl)phenyl)-4,5-dihydro-1H-thiazolo[3,4-a]quinazoline-3-carboxamide)

yellow solid (80 mg, 20%). 1H NMR (400 MHz, DMSO-d₆) δ 12.25 (s, 1H), 10.68 (d, J=9.2 Hz, 1H), 8.23 (d, J=2.3 Hz, 1H), 8.15 (s, 1H), 7.90 (dd, J=9.2, 3.1 Hz, 1H), 7.68 (d, J=8.4 Hz, 1H), 7.58 (t, J=7.6 Hz, 1H), 7.38 (d, J=7.6 Hz, 1H)

### 16) Compound 209 (7-bromo-5-oxo-1-thioxo-N-(3-(trifluoromethoxy)phenyl)-4,5-dihydro-1H-thiazolo[3,4-a]quinazoline-3-carboxamide)

yellow solid (57 mg, 15%). 1H NMR (400 MHz, DMSO-d₆) δ 10.57 (d, J=9.2 Hz, 1H), 10.52 (s, 1H), 8.25 (d, J=2.7 Hz, 1H), 8.11 (dd, J=9.2, 2.7 Hz, 1H), 7.80 (s, 1H), 7.66 (d, J=8.0 Hz, 1H), 7.50 (t, J=8.2 Hz, 1H), 7.13 (d, J=8.0 Hz, 1H)

### 17) Compound 210 (N-(3,5-bis(trifluoromethyl)phenyl)-7-bromo-5-oxo-1-thioxo-4,5-dihydro-1H-thiazolo[3,4-a]quinazoline-3-carboxamide)

yellow solid (19 mg, 5%). 1H NMR (400 MHz, methanol-d₄) δ 10.74 (d, J=9.2 Hz, 1H), 8.54 (s, 2H), 8.34 (d, J=2.4 Hz, 1H), 7.76 (dd, J=9.3, 2.6 Hz, 1H), 7.53 (s, 1H)

### Example 1. BK_{Ca} Activation Effects of Compounds TTQC-1 to TTQC-34

### 1.1 Materials and Methods

### 1) Materials

The G-protein bound receptor targeting chemical library (9,938 compounds) was provided by the KRICT Compound Bank (Chemical Bank of Korea Research Institute of Chemical Technology, Daejeon, South Korea). Dimethyl sulfoxide (DMSO) (≥99.7%) was purchased from Sigma Aldrich (St. Louis, Missouri, USA), and solifenacin succinate (≥98%) was purchased from Merck (Darmstadt, Hessen, Germany). PEG 400 (polyethylene glycol 400) was purchased from Samcheon Chemical (Seoul, Korea).

TTQC-1 for use in the in vivo study was synthesized in the laboratory of Gwangju Institute of Science and Technology. Reagents used in this study were purchased from Sigma Aldrich, TCI (Tokyo, Japan) and Alfa Aesar (Haverhill, MA, USA), and used without further purification. Thin layer chromatography was performed using a glass plate pre-coated with silica gel (silica gel 60, F-254, 0.25 nm) purchased from Merck. Identification of substances separated by thin layer chromatography was confirmed using a UV lamp (254 nm, 365 nm). For purification of the reactant, the column was packed using silica gel grade 9385 (230-400 mesh; Merck). To confirm the structure of the synthesized compounds, 1H NMR spectrum was performed on a JEOL JNM-ECS400 spectrometer at 400 MHz.

### 2) Cell Culture and Cell-Based Fluorescence Assay

AD 293 cells, which are derivatives of the commonly used HEK 293 cell line and stably express hyperactive mutant BK_{Ca} channels (hSlo G803D/N806K) to sensitively detect the effect (Lee et al., 2013), were cultured in Dulbecco' modified Eagle medium (Hyclone, Logan, Utah, UK) supplemented with 10% fetal bovine serum, and selected with 1 mg/mL Geneticin (Gibco, Amarillo, TX, USA). 20,000 cells per well were seeded on a 96-well clear-bottom black plate (Corning, New York, NY, USA) coated with poly-D-lysine (Gibco). BKCa channel activity was measured using the cell-based fluorescence assay, FluxOR Potassium Ion Channel Assay (Thermo Fisher Scientific, Waltham, MA, USA). Experimental procedures were performed according to the manufacturer's instructions. The cells were treated with test compounds dissolved in assay buffer for 15 minutes. Fluorescence was measured using a FlexStation3 multimode microplate reader (Molecular Devices, Silicon Valley, CA, USA) and SoftMax Pro (Molecular Devices). Excitation and emission wavelengths were set to 485 nm and 528 nm, respectively. Fluorescence signals were acquired every 2 seconds for 120 seconds and normalized by the relative fluorescence unit (RFU). The efficacy of a channel activator was determined by analyzing changes (ΔRFU) in the RFU for 80 seconds after channel stimulation.

### 3) Synthesis and Injection of Complementary RNA

The complete coding sequence (CDS) of each of rat KCNMA (rSlo α) (GenBank AF135265.1), KCNMB1 (rSlo 01) (GenBank FJ154955.1) and KCNMB4 (rSlo β4) (GenBank AY028605) was subcloned into pNBC2.0 or pNBC2.0. These vectors were designed to be expressed in Xenopus oocytes (Ha, T. S., Lim, H. H., Lee, G. E., Kim, Y. C., & Park, C. S. (2006). Electrophysiological characterization of benzofuroindole-induced potentiation of large-conductance Ca2+-activated K+ channels. Mol Pharmacol, 69(3), 1007-1014). The plasmid was linearized by NotI and complementary RNA (cRNA) was synthesized using the Ambion T7 mMESSAGE mMACHINE kit (Invitrogen, Carlsbad, CA, USA) according to the manufacturer's instructions. Xenopus laevis (KXRCR000001) was obtained from the Korea Xenopus Resource Center for Research (Chuncheon-si, Gangwon-do, Korea). Stage V-VI oocytes were surgically extracted from the ovarian lobes of X. laevis. A follicular cell layer was removed by culturing in Ca²⁺-free oocyte Ringer's medium (86 mM NaCl, 1.5 mM KCl, 2 mM MgCl₂ and 10 mM HEPES, pH 7.6) containing collagenase for 1.5 hours at room temperature. Oocytes without follicular layer were washed with ND-96 medium (96 mM NaCl, 2 mM KCl, 1.8 mM CaCl₂), 1 mM MgCl₂, 5 mM HEPES, 50 g/mL gentamicin, pH 7.6). The prepared oocytes were stabilized overnight at 18° C. cRNA was injected into each oocyte (50 ng per oocyte) using a microdispenser (Drummond Scientific, Broomall, PA, USA). For co-expression of the β subunits, cRNA was mixed at a molar ratio of 1:12 (α:β) to induce sufficient co-assembly of the R subunits. After injection of cRNA, the oocytes were cultured in ND-96 medium for 1-3 days at 18° C. Before recording macroscopic currents, vitelline membranes of the oocytes were completely removed using fine forceps.

### 4) Macroscopic Current

All macroscopic current recordings in the BK_{Ca} channels were performed using the gigaohm-seal patch-clamp method in an outside-out configuration (Ha et al., 2006). A patch pipette was made of borosilicate glass (WPI, Sarasota, FL, USA) and fire-polished by a resistance of 3-5 MQ. Ion currents were amplified using an Axopatch 200B amplifier (Molecular Devices). The currents were low-pass filtered at 1 kHz using a four-pole Bessel filter and digitized at a rate of 10 or 20 points/ms using a Digidata 1200A interface (Molecular Devices). The BK_{Ca} channels were activated by repeated voltage clamps at +100 mV or by voltage clamp pulses ranging from −80 to +200 mV with an increment of 10 mV. The resting potential was maintained at −100 mV. The recording solution contained 116 mM KOH, 4 mM KCl, 10 mM HEPES, and 5 mM EGTA (pH 7.2). The intracellular solution contained 3 μM Ca²⁺ in the recording solution (pH 7.0). MaxChelator program was used to calculate the total amount of Ca²⁺ to be added to the intracellular solution for obtaining free Ca²⁺ at a concentration of 3 μM.

### 5) Electrophysiological Data Analysis

The commercial software Clampex 8.0 (Molecular Device) and Origin 9.1 (OriginLab Corp., Northampton, MA, USA) were used for data analysis and fitting of the macroscopic currents. A conductance-voltage (G-V) curve obtained from the external current was fitted by the Boltzmann equation [y=(Gₘₐₓ−Gₘᵢₙ)/{1+exp[(V_{1/2}−x)/k]}+Gₘᵢₙ]. Wherein k is RT/zF, and wherein R is a gas constant, T is a temperature, F is the Faraday's constant, and z is a gating charge. Gₘₐₓ is the maximum conductivity and V_{1/2} is a voltage for half maximum conductivity. The concentration-dependent V_{1/2} shift was fitted by the Hill equation [y=AVi/2,max x\hat{ }n/(EC50\hat{ }n+x\hat{ }n)]. Wherein ΔV_{1/2,max} is a constant. EC₅₀ is half the apparent maximum effective concentration and n is the Hill coefficient. By definition, the apparent dissociation constant K_{D} was obtained by EC₅₀\hat{ }n. The association time constant (r association) was obtained by fitting the exponential equation [y=A exp(−x/τ)+C]. Wherein A and C are constants and τ is the time to reach (1−1/e) ( [symbol] 63%) of the maximum current level. The dissociation time constant (τ dissociation) was obtained by fitting a double exponential equation [y=Afast exp(−x/τ fast)+Aslow exp(−x/τ slow)+C]. To obtain gating kinetics of the BK_{Ca} channels, the activation (t activation) or deactivation (r deactivation) time constant is calculated by fitting the exponential equation [y=A exp(−x/τ)+C] from the outward current or tail current, respectively.

### 6) In Vivo Model of OAB

Spontaneously hypertensive rats (SHR) were used as an animal model of OAB because they urinate significantly more frequently than the normotensive control, Wistar-Kyoto rats (WKY). Male rats with body weights of 300 to 350 g were deprived of food and water overnight before the experiment, and then were randomly divided into a drug administration group and a control group. All animals were provided free access to water for 2 hours, and then 10 mL/kg of vehicle solution [DMSO:PEG400:distilled water=5:40:55 (v/v)] as a test substance were orally administered. After oral administration of the test substance, the rats were placed in a metabolic cage and the number of urinations and urine volume were measured for 3 hours.

### 7) Statistics

Data are represented by average±SEM for the indicated number of independently performed experiments. Statistical significance (p<0.05) was assessed by Student's t-test for cell-based fluorescence assay and electrophysiological experiment. For in vivo experiments, one-way ANOVA was performed and Dunnett's test was used as a post hoc analysis to assess statistical significance.

### 1.2. Results

### 1) Screening and Discovery of Novel BK_{Ca} Channel Activators Using Cell-Based Screening

A library of compounds was screened for determining their activation effects on the BK_{Ca} channels using the cell-based fluorescence assay. A group of compounds with similar structures that have significant activation effects on the BK_{Ca} channels has been identified. These compounds increased fluorescence by 1.2-4.1 times at 5 μM compared to vehicle (p<0.05) (FIG. 1A). The previously reported BK_{Ca} channel activator LDD175 (Gormemis, A. E., Ha, T. S., Im, I., Jung, K. Y., Lee, J. Y., Park, C. S., & Kim, Y. C. (2005). Benzofuroindole analogues as potent BK(Ca) channel openers. Chembiochem, 6(10), 1745-1748.) was included as a positive control in this analysis. The chemical structure of the identified compound is characterized by the presence of a quinazoline ring directly linked to an amide bond and various functional groups. The basic structures of these TTQC (thioxo-thiazoloquinazoline-carboxamide) compounds are shown in FIG. 1B and listed in Table 1 along with molecular weight and activity. The TTQC-1 compound corresponding to the structure c showed relatively higher activity than other groups. Compound TTQC-1 (7-bromo-N-(3-methylphenyl)-5-oxo-1-thioxo-4,5-dihydro[1,3]thiazolo[3,4-a]quinazoline-3-carboxamide) containing a bromo group at R₂ position and a methyl group at R₄ position in the structure c in FIG. 1B was more effective in activating the BK_{Ca} channels than LDD175 (FIG. 1A and Table 1).

Table 1 below shows information on each substituent included in the structures of the compounds in FIG. 1B.

TABLE 1

| | Back | | | | | | | MW | |
|---|---|---|---|---|---|---|---|---|---|
| Compound | bone | R1 | R2 | R3 | R4 | R5 | R6 | (g/mole) | Activity |
| TTQC-1 | c | H | Br | H | CH₃ | H | H | 446.34 | 1.06 ± |
| | | | | | | | | | 0.037 |
| LDD175 | NA | NA | NA | NA | NA | NA | NA | 353.68 | 1.00 ± |
| | | | | | | | | | 0.058 |
| TTQC-2 | c | COOCH₃ | H | H | F | H | H | 429.44 | 0.99 ± |
| | | | | | | | | | 0.016 |
| TTQC-3 | c | Cl | H | H | H | COOCH₂CH₃ | H | 459.92 | 0.96 ± |
| | | | | | | | | | 0.095 |
| TTQC-4 | c | Cl | H | CH₂CH₃ | H | H | H | 415.91 | 0.95 ± |
| | | | | | | | | | 0.044 |
| TTQC-5 | c | H | C1 | OCH₃ | H | H | H | 417.88 | 0.90 ± |
| | | | | | | | | | 0.039 |
| TTQC-6 | a | H | Br | CH(CH₃)₂ | NA | NA | NA | 398.29 | 0.90 ± |
| | | | | | | | | | 0.007 |
| TTQC-7 | c | H | CH₃ | CH₃ | H | H | Cl | 415.91 | 0.89 ± |
| | | | | | | | | | 0.020 |
| TTQC-8 | e | COOCH₃ | H | Cl | H | H | NA | 445.89 | 0.84 ± |
| | | | | | | | | | 0.040 |
| TTQC-9 | c | H | Cl | CH₃ | H | CH₃ | H | 415.91 | 0.84 ± |
| | | | | | | | | | 0.033 |
| TTQC- | c | H | Br | CH₃ | H | CH₃ | H | 460.36 | 0.83 ± |
| 10 | | | | | | | | | 0.004 |
| TTQC- | c | H | CH₃ | CH₃ | H | H | H | 381.47 | 0.82 ± |
| 11 | | | | | | | | | 0.023 |
| TTQC- | d | NA | NA | CH₃ | H | H | Cl | 445.89 | 0.81 ± |
| 12 | | | | | | | | | 0.130 |
| TTQC- | c | COOCH₃ | H | F | H | F | H | 447.43 | 0.79 ± |
| 13 | | | | | | | | | 0.047 |
| TTQC- | e | H | Br | H | H | CH₃ | NA | 460.36 | 0.76 ± |
| 14 | | | | | | | | | 0.022 |
| TTQC- | c | H | Br | OCH₃ | H | H | OCH₃ | 492.36 | 0.71 ± |
| 15 | | | | | | | | | 0.038 |
| TTQC- | a | H | Br | C₅H₁₀O | NA | NA | NA | 440.33 | 0.64 ± |
| 16 | | | | | | | | | 0.023 |
| TTQC- | c | OCH₃ | OCH₃ | CH₃ | H | H | Cl | 461.94 | 0.64 ± |
| 17 | | | | | | | | | 0.033 |
| TTQC- | c | H | CH₃ | OCH₃ | H | H | OCH₃ | 427.49 | 0.63 ± |
| 18 | | | | | | | | | 0.054 |
| TTQC- | d | NA | NA | CH₂CH₃ | H | H | H | 425.48 | 0.62 ± |
| 19 | | | | | | | | | 0.021 |
| TTQC- | e | H | Br | Cl | H | H | NA | 480.78 | 0.62 ± |
| 20 | | | | | | | | | 0.003 |
| TTQC- | e | H | Cl | Cl | H | H | NA | 436.33 | 0.60 ± |
| 21 | | | | | | | | | 0.012 |
| TTQC- | c | OCH₃ | OCH₃ | H | H | H | H | 413.47 | 0.57 ± |
| 22 | | | | | | | | | 0.022 |
| TTQC- | a | Cl | H | C₅H₉ | NA | NA | NA | 379.88 | 0.55 ± |
| 23 | | | | | | | | | 0.019 |
| TTQC- | d | NA | NA | OCH₃ | H | H | H | 427.45 | 0.51 ± |
| 24 | | | | | | | | | 0.019 |
| TTQC- | a | Cl | H | CH₃ | NA | NA | NA | 325.79 | 0.50 ± |
| 25 | | | | | | | | | 0.009 |
| TTQC- | c | Cl | H | F | H | Br | H | 484.74 | 0.49 ± |
| 26 | | | | | | | | | 0.023 |
| TTQC- | a | H | Cl | C₅H₁₀O | NA | NA | NA | 395.88 | 0.47 ± |
| 27 | | | | | | | | | 0.010 |
| TTQC- | c | H | Cl | OCH₂CH₃ | H | H | H | 476.36 | 0.45 ± |
| 28 | | | | | | | | | 0.026 |
| TTQC- | e | Cl | H | H | OCH₃ | H | NA | 431.91 | 0.45 ± |
| 29 | | | | | | | | | 0.010 |
| TTQC- | b | H | Br | NA | NA | NA | NA | 424.33 | 0.37 ± |
| 30 | | | | | | | | | 0.007 |
| TTQC- | c | H | CH₃ | CH₃ | H | CH₃ | H | 395.5 | 0.34 ± |
| 31 | | | | | | | | | 0.007 |
| TTQC- | c | H | Br | H | H | CH₂CH₃ | H | 460.36 | 0.34 ± |
| 32 | | | | | | | | | 0.011 |
| TTQC- | e | Cl | H | H | H | OCH₃ | NA | 431.91 | 0.34 ± |
| 33 | | | | | | | | | 0.009 |
| TTQC- | c | Cl | H | OCH₃ | H | H | OCH3 | 447.91 | 0.31 ± |
| 34 | | | | | | | | | 0.008 |
| Vehicle | NA | NA | NA | NA | NA | NA | NA | NA | 0.26 ± |
| | | | | | | | | | 0.061 |

### 2) Comparison of Activation Effects Between TTQC-1 and Other Chemical Activators on BKCa Channels

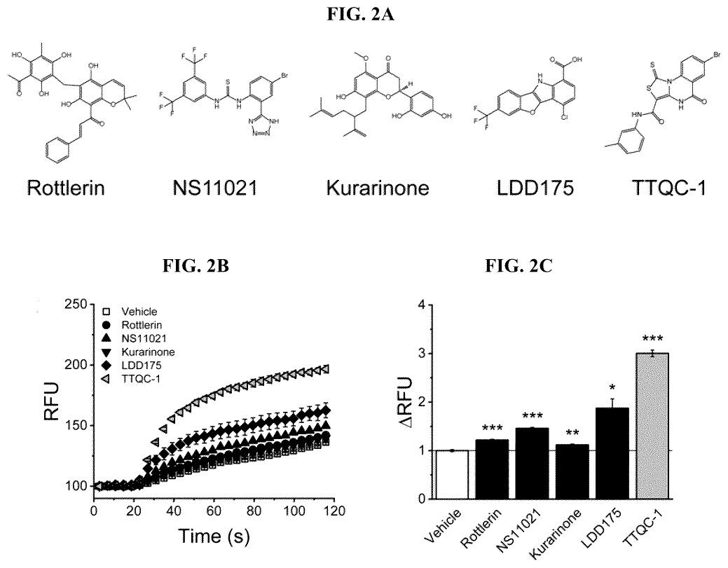

Next, the activation effect of TTQC-1 at a single concentration was compared with that of several well-known BK_{Ca} channel activators including rottlerin, NS1102T, kurarinone and LDD175 (FIG. 2A). These compounds showed significant activation effects on the BK_{Ca} channels, but TTQC-1 showed the most potent effect at 3 μM in the in vitro assay. The activation effect of TTQC-1 on the BK_{Ca} channel was 1.6 times greater than that of LDD175 (FIGS. 2B and 2C).

### 3) Effect of BKCa Channel Inhibitors Paxillin and Iberiotoxin on TTQC-1 Activity

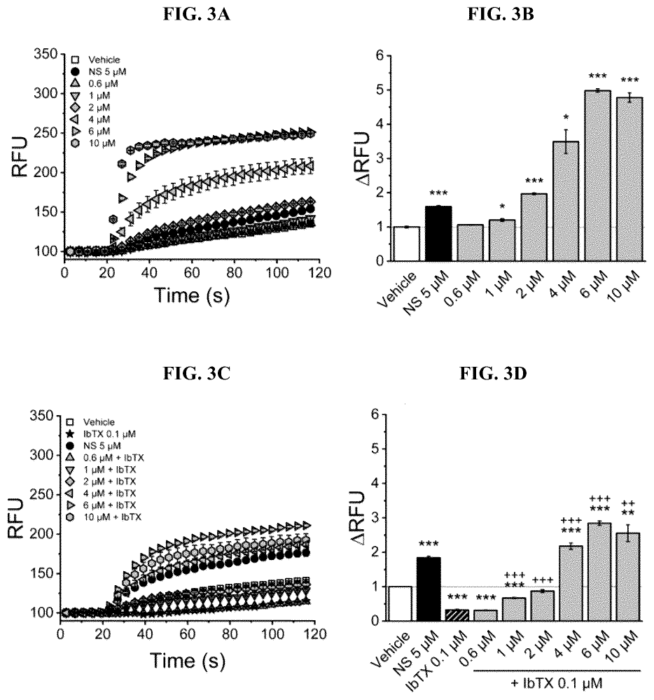

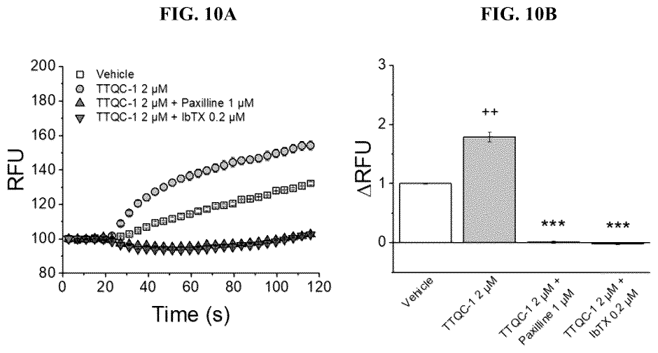

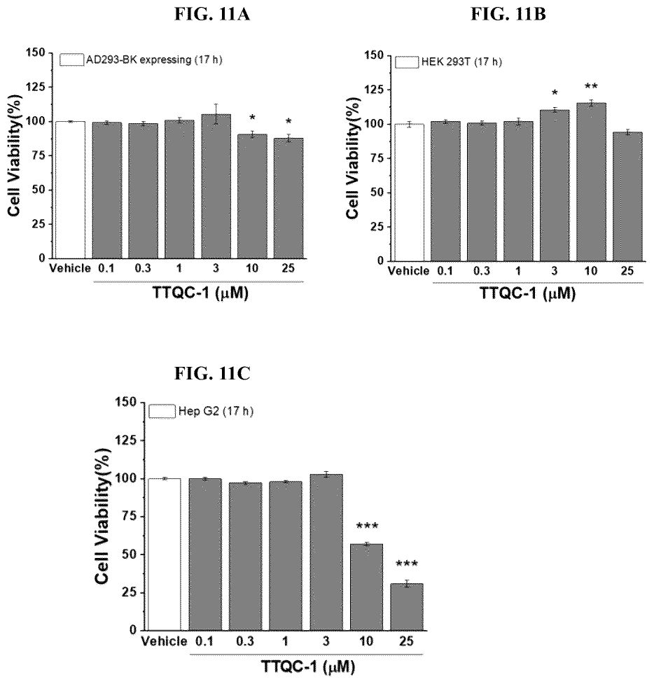

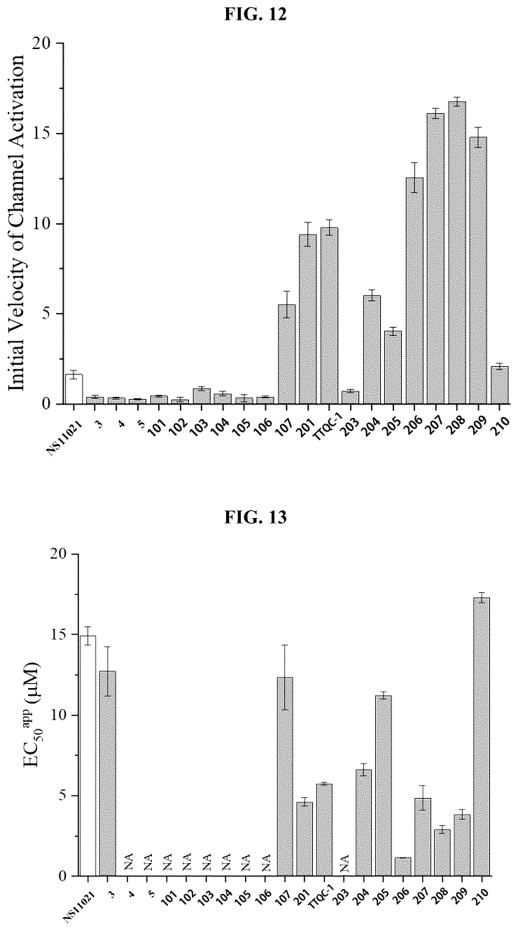

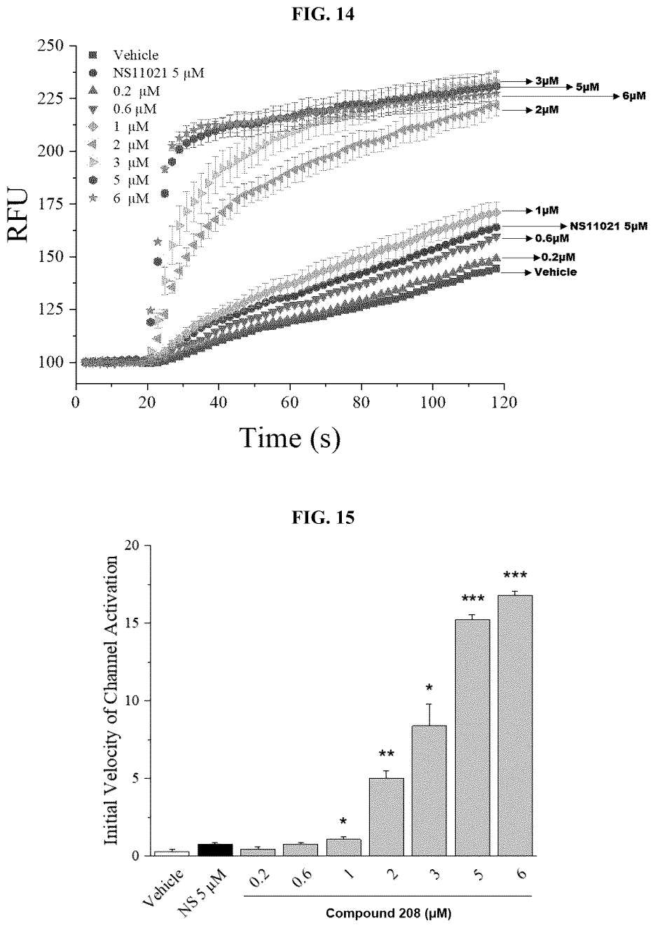

To determine whether the increase in fluorescence mediated by TTQC-1 was caused by Tl⁺ flux through the BK_{Ca} channels, the present inventors investigated the effect of TTQC-1 in the presence of two BK_{Ca} channel inhibitors, paxillin and iberiotoxin. The increase in fluorescence induced by 2 μM TTQC-1 was removed by 1 μM paxillin and 0.1 μM iberiotoxin (FIGS. 10A and 10B). Next, when treating or untreating with 0.1 μM iberiotoxin, the activation effects of TTQC-1 at various concentrations were investigated. TTQC-1 concentration-dependently increased Tl⁺ flux and effect were saturated at 6 μM in the in vitro assay (FIGS. 3A and 3B). The effect of TTQC-1 was 4.98±0.05 times greater than that of the vehicle at 6 μM. Iberiotoxin eliminated the TTQC-1 activation effect at concentrations below 2 μM, but TTQC-1 at concentrations above 4 μM activated the BK_{Ca} channels in the presence of 0.1 μM iberiotoxin (FIGS. 3C and 3D). These results show that the enhancing effect of TTQC-1 is non-competitively inhibited by iberiotoxin, and thus that TTQC-1 and iberiotoxin do not compete for the same binding site of the channel.

### 4) Effect of TTQC-1 on BK_{Ca} Channel Macroscopic Current

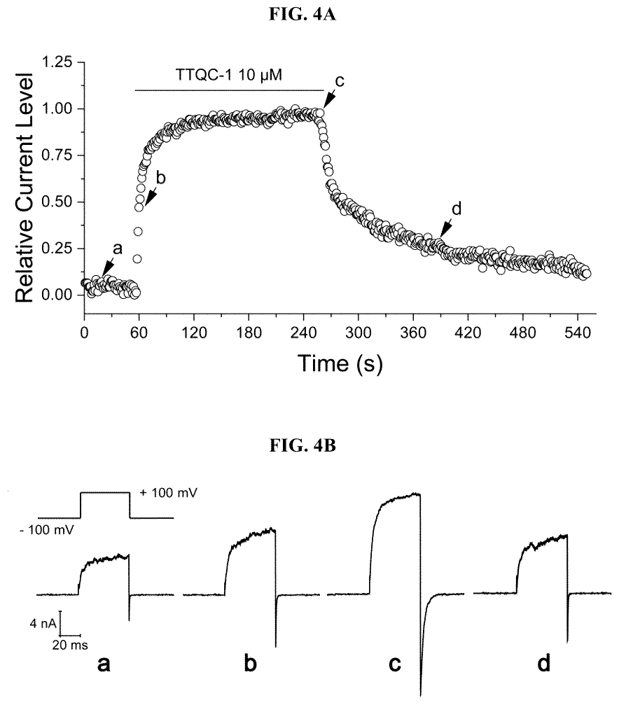

Based on the results of the in vitro assay, the direct effect of TTQC-1 on BK_{Ca} channel currents was verified using electrophysiology. The BK_{Ca} channels were expressed in Xenopus oocytes and outside-out incision portions of the membrane were harvested. TTQC-1 was injected into an extracellular side of the BK_{Ca} channel and ionic currents were measured upon voltage stimulation. Perfusion of 10 μM TTQC-1 rapidly increased the current, and perfusion of bath solution eliminated the effect of TTQC-1 (FIG. 4A). The association time constant (i association) related to the time to reach about 63.2% of the activated channel was fitted with a single exponential equation and estimated to be 11.7±1.77 seconds. The dissociation time constant (i dissociation) related to the time to reach about 63.2% of the deactivated channel was fitted with a double exponential equation, and the measured time constants were 7.8±1.10 seconds (A_{fast} 0.49±0.064) for the fast component and 136.2±11.24 seconds (A_{slow} 0.53±0.054) for the slow component, respectively. FIG. 4B shows representative current traces at each perfusion state (panels a-d in FIG. 4A).

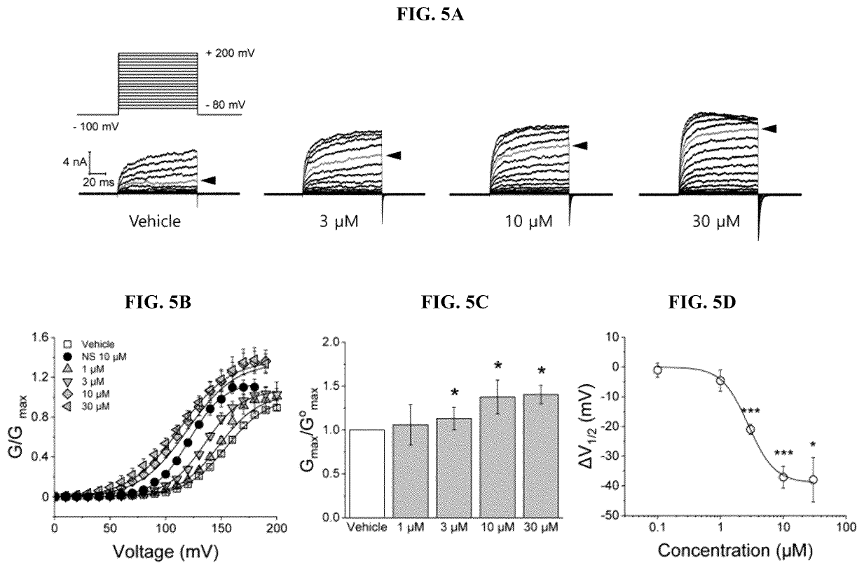

Next, since the BK_{Ca} channels were activated by depolarizing potentials, the effects of TTQC-1 on the relationship between channel conductance and membrane voltage (G-V relationship) were investigated. The G-V relationship was fitted by the Boltzmann equation, and then V_{1/2} and Gₘₐₓ were calculated. TTQC-1 increased the current in a concentration-dependent manner and shifted the G-V curve to a negative potential (FIGS. 5A and 5B). Quantitatively, 30 μM TTQC-1 increased the maximum conductance (Gₘₐₓ) by 1.4±0.11 times compared to the vehicle treated channel (G^{o}ₘₐₓ) (FIG. 5C). These results indicate that more channels are opened at the same voltage stimulation in the presence of TTQC-1. In addition, 30 μM TTQC-1 reduced the half-activation voltage (V_{1/2}) to 37.9±7.50 mV (FIG. 5D), which indicates that the channel was opened at a lower voltage in the presence of TTQC-1. The V_{1/2} shift was fitted by the Hill equation and the apparent EC₅₀ value was calculated to be 2.8 μM. The Hill coefficient n was 2.0, which indicates that positive cooperativity occurred while TTQC-1 binds to the channel.

### 5) Effect of TTQC-1 on BK_{Ca} Channel Gating

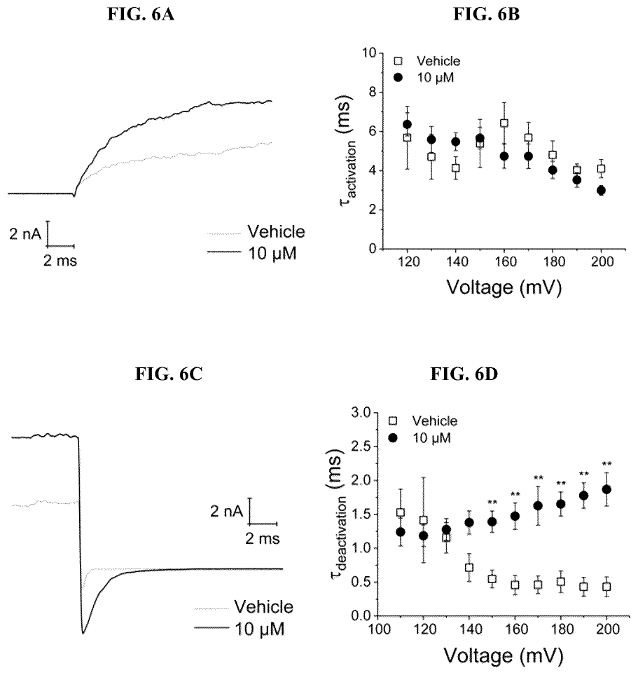

The channel opening time and channel closing time were analyzed by fitting the outward current and tail current to the exponential equation, respectively. The channel activation time constant (τ activation) is a constant of the time to reach about 63% of the maximum outward current, and the channel deactivation time constant (τ deactivation) is a constant of the time for about 63% of the maximum tail current to disappear. The BK_{Ca} channels began to open at pulses of at least 100 mV. When the BK_{Ca} channels were perfused with 10 μM TTQC-1, τ activation did not show significant changes within the tested voltage range (FIGS. 6A and 6B). However, τ deactivation was significantly increased after +150 mV, up to 5.3 times at +200 mV (FIGS. 6C and 6D). These results indicate that TTQC-1 did not affect the channel opening but delayed the channel closing.

### 6) Effect of TTQC-1 on Macroscopic Current of the BKCa Channel Co-Expressed with Beta Subunit

The BK_{Ca} channels are expressed and bound with auxiliary subunits such as β subunits. Four subtypes of the β subunits may change the macroscopic dynamics, apparent calcium and voltage sensitivity of the channel in different ways.

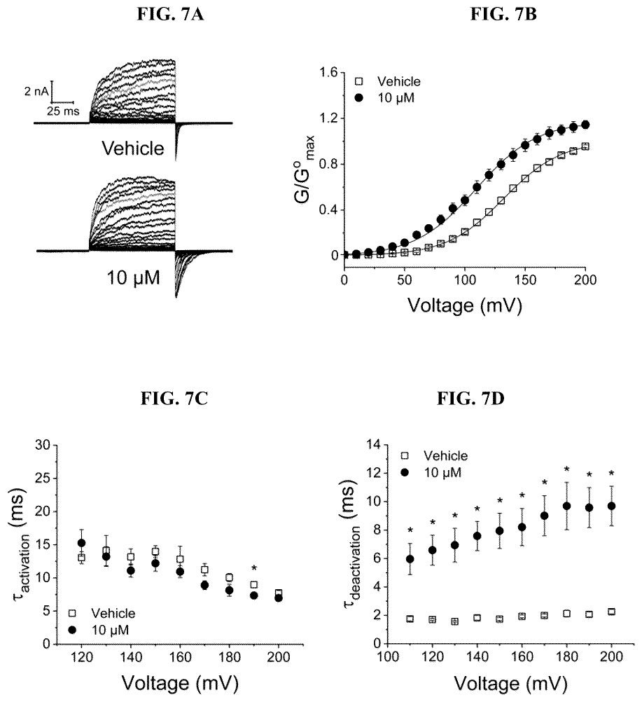

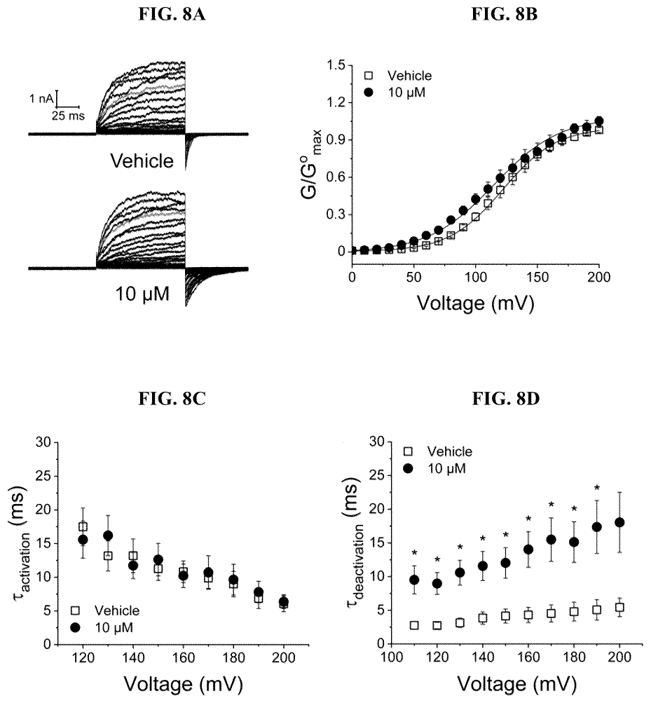

First, the present inventors tested the subunit-dependent effects of TTQC-1 in the presence of β1 and β4 subunits, which are highly expressed in the bladder. The shape of the ionic currents was changed when the β1 and β4 subunits were co-expressed with the BK_{Ca} channels. When treating the BK_{Ca} channels co-expressed with the β subunits with 10 μM TTQC-1, the shape of the macroscopic currents was further changed (FIGS. 7A and 8A). TTQC-1 shifted the G-V curve of BK_{Ca} co-expressed with the β1 subunit to the left by 23.0±2.67 mV (FIG. 7B). TTQC-1 changed the τ activation only at 190 mV (FIG. 7C), but significantly increased the τ deactivation up to 4.6 times at 180 mV (FIG. 7D). When the BK_{Ca} channels were co-expressed with the β4 subunits, TTQC-1 shifted the G-V curve to the left by 7.12±0.65 mV (FIG. 8B). TTQC-1 did not significantly affect the τ activation (FIG. 8C), but the τ deactivation was significantly increased up to 3.7 times at 170 mV (FIG. 8D). These results show that although the shift of the G-V curve was dependent on co-expression of the β1 and β4 subunits, the time constants of channel gating, τ activation and τ deactivation were not unduly affected by the presence of the auxiliary subunits.

### 7) Effect of TTQC-1 on Urination in Hypertensive Rats

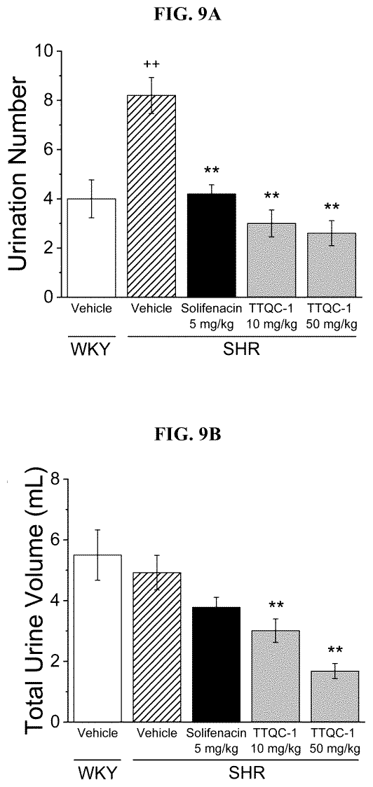

The in vivo efficacy of TTQC-1 was confirmed using an animal model of OAB. Spontaneously hypertensive rats (SHR) urinate frequently, which is a typical symptom of OAB. The synthesized compounds were administered orally and urinary behavior was recorded for 3 hours. Normally urinating Wistar Kyoto rats (WKY) were used as the control. The in vivo efficacy of TTQC-1 was compared with solifenacin succinate, a commercially available OAB drug targeting an M3 muscarinic acetylcholine receptor. Spontaneously hypertensive rats (SHR) urinated 2.1 times more frequently than Wistar Kyoto rats (WKY). As a result of administering 10 mg/kg TTQC-1 to SHR, the number of urinary events was significantly reduced from 8.2 to 3.0 similar to the number observed in WKY. When 5 mg/kg of solifenacin was administered to SHR, the number of urinations was decreased from 8.2 to 4.2. In addition, the total urine volume was significantly reduced by administration of TTQC-1 (FIGS. 9A and 9B).

### Example 2. Effects of Compounds 101-107 and 201-210 on BK_{Ca} Channel Activation and Overactive Bladder Treatment

### 2.1. Evaluation of Compounds 101-107 and 201-210 on BK_{Ca} Channel Activation Ability Using Cell-Based Fluorescence Assay

Activation effects of the synthesized compounds on BK_{Ca} channels were evaluated using cell-based fluorescence assay. NS11021 was used as the positive control. The effects were determined based on the initial rate (vi) of channel activation and the apparent EC₅₀ value obtained from fitting data of the dose-response curve. Here, Vi means change values for 4 seconds at the start of channel opening (vi={RFU_{(t=24 s)}−RFU_{(t=20 s)}}/4 s). In addition, activity was measured by measuring ΔRFU (relative fluorescence unit) at 6 μM (Tables 1-3). ΔRFU means the change values for 80 seconds after channel start (ΔRFU={RFU_{(t=100 s)}−RFU_{(t=20 s)}}/{RFU_{(t=100 s)}^{veh}−RFU_{(t=20 s)}^{veh}}), and maximum activity can be confirmed.

Compound 3 showed moderate BK_{Ca} channel activation (vi=0.38, EC₅₀=12.72 μM and ΔRFU=1.38 at 6 μM). Compounds 4, 5 and 101 were synthesized and subjected to evaluation for BK_{Ca} efficacy (channel activation). Results thereof are shown in Table 2 below.

TABLE 2

| | | Initial | Apparent | Channel |
|---|---|---|---|---|
| | | velocity of | EC₅₀ | activity |
| | | channel | based | increase |
| | | activation | on vᵢ | for |
| | | for 4 s (vᵢ) | (μM) | 80 s |
| | | at 6 μM | Apparent | (ARFU) at |
| Compound | Structure | (sec⁻¹) | EC₅₀ | 6 μM |
| 3 | 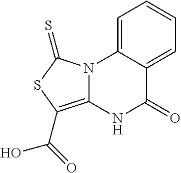 [SMILES: O=C(O)c1sc(=S)n2c1[nH]c(=O)c1ccccc12] | 0.38 | 12.72 | 1.38 |
| 4 | 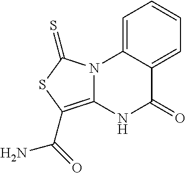 [SMILES: NC(=O)c1sc(=S)n2c1[nH]c(=O)c1ccccc12] | 0.32 | NA | 0.96 |
| 5 | 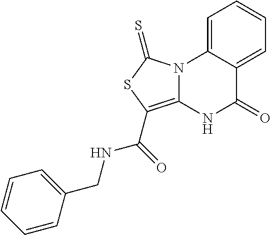 [SMILES: O=C(NCc1ccccc1)c1sc(=S)n2c1[nH]c(=O)c1ccccc12] | 0.26 | NA | 1.10 |
| 101 | 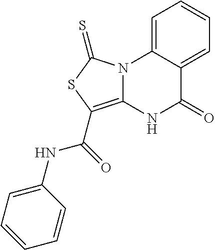 [SMILES: O=C(Nc1ccccc1)c1sc(=S)n2c1[nH]c(=O)c1ccccc12] | 0.42 | NA | 1.03 |
| NS11021 | 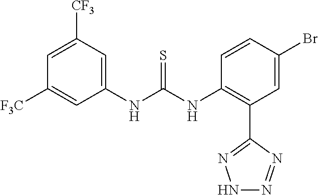 [SMILES: FC(F)(F)c1cc(NC(=S)Nc2ccc(Br)cc2-c2nn[nH]n2)cc(C(F)(F)F)c1] | 1.63 | 14.92 | 1.86 |

Compound 101 increased channel activation (vi=0.42 at 6 μM, ΔRFU=1.03 at 6 μM). Compounds 4 and 5 showed lower efficacy (vi=0.32 and 0.26 at 6 μM, ΔRFU=0.96 and 1.10 at 6 μM, respectively). Based on these results, anilide analogues of Compound 101 were investigated.

To confirm the substitution effect on 1-thioxo-1H-thiazolo[3,4-a]quinazolin-5(4H)-one, activation effects of compounds having an electron-donating group (EDG) such as a methyl group (Compound 102) at position 8 of the phenyl ring, or compounds having an electron-withdrawing group (EWG) functional group such as a chloro group (Compound 103) or bromo group (Compound 104) on BK_{Ca} channels were evaluated. In addition, activation effects of compounds, into which methyl (Compound 105), chloro (Compound 106), and bromo (Compound 107) groups are introduced at position 7, on BK_{Ca} channel were evaluated. As shown in Table 3, Compounds 102, 103, 104, 105 and 106 showed a slight increase in activity (vi=0.23-0.84 at 6 μM, ΔRFU=1.03-1.56 at 6 μM). Compound 107 showed significant improvement in channel opening activity with vi=5.51, EC₅₀=12.33 μM and ΔRFU 3.83 at 6 μM. That is, it can be seen that a 7-bromo substituent of 1-thioxo-1H-thiazolo[3,4-a]quinazolin-5(4H)-one showed significant improvement in BK_{Ca} channel opening activity.

TABLE 3

| | | Initial | | Channel |
|---|---|---|---|---|
| | | velocity of | | activity |
| | | channel | Apparent | increase |
| | | activation | EC₅₀ | for |
| | | for 4 s (vᵢ) | based | 80 s |
| | | at 6 μM | on vᵢ | (ΔRFU) at |
| Compound | Structure | (sec⁻¹) | (μM) | 6 μM |
| 101 | 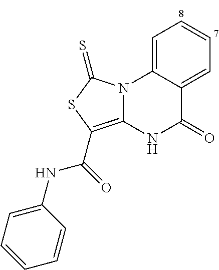 [SMILES: O=C(Nc1ccccc1)c1sc(=S)n2c1[nH]c(=O)c1ccccc12] | 0.42 | NA | 1.03 |
| 102 | 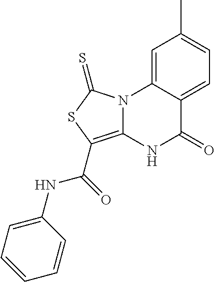 [SMILES: Cc1ccc2c(=O)[nH]c3c(C(=O)Nc4ccccc4)sc(=S)n3c2c1] | 0.23 | NA | 1.06 |
| 103 | 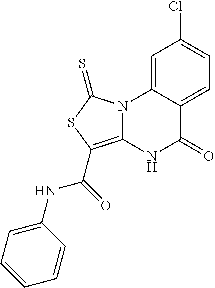 [SMILES: O=C(Nc1ccccc1)c1sc(=S)n2c1[nH]c(=O)c1ccc(Cl)cc12] | 0.84 | NA | 1.56 |
| 104 | 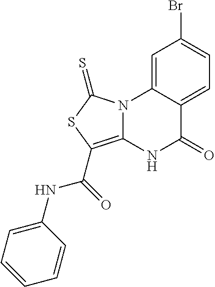 [SMILES: O=C(Nc1ccccc1)c1sc(=S)n2c1[nH]c(=O)c1ccc(Br)cc12] | 0.56 | NA | 1.25 |
| 105 | 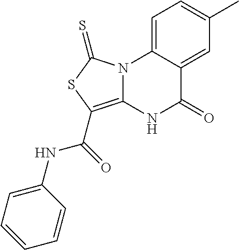 [SMILES: Cc1ccc2c(c1)c(=O)[nH]c1c(C(=O)Nc3ccccc3)sc(=S)n12] | 0.32 | NA | 1.16 |
| 106 | 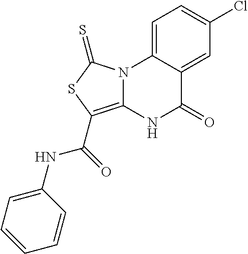 [SMILES: O=C(Nc1ccccc1)c1sc(=S)n2c1[nH]c(=O)c1cc(Cl)ccc12] | 0.38 | NA | 1.18 |
| 107 | 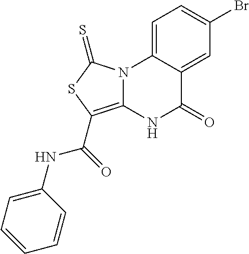 [SMILES: O=C(Nc1ccccc1)c1sc(=S)n2c1[nH]c(=O)c1cc(Br)ccc12] | 5.51 | 12.33 | 3.83 |
| NS11021 | 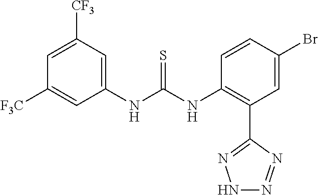 [SMILES: FC(F)(F)c1cc(NC(=S)Nc2ccc(Br)cc2-c2nn[nH]n2)cc(C(F)(F)F)c1] | 1.63 | 14.92 | 1.86 |

Next, several analogues, in which anilide was changed from Compound 107 that is the 7-bromo substituent in the tricyclic system, were synthesized and subjected to evaluation for BK_{Ca} activity (see Table 4). Compound 201, in which phenyl of Compound 107 was substituted with ortho-methylphenyl (2-methylphenyl), showed an increase in the channel activity (vi=9.40, EC₅₀=4.60 μM at 6 μM, ΔRFU=3.68 at 6 μM). In addition, meta-methyl substituted Compound 202 (vi=9.77 at 6 μM, EC₅₀=5.74 μM, ΔRFU=4.98 at 6 μM) was more potent than ortho-methyl substituted compound (201). When replacing the meta-methyl in compound TTQC-1 with meta-methoxy (Compound 204) and meta-hydroxy (Compound 205), activation was reduced (204: vi=6.00, EC₅₀=6.60 μM at 6 μM, ΔRFU=3.35 at 6 μM; 205: vi=4.03, EC₅₀=11.22 μM at 6 μM, ΔRFU=4.88 at 6 μM). Halogen-substituted Compound 206 showed significant improvement in the BK_{Ca} activity. Among the halogen-substituted compounds, meta-trifluoromethyl Compound 208 showed the highest channel activity titer with vi=16.77 and EC₅₀=2.89 μM at 6 μM. Compound 209 showed slightly lower activity than Compound 208 with vi=14.78, EC₅₀=3.82 at 6 μM, and Compound 210 having a 3,5-bis(trifluoromethyl) substituent showed lower activity with vi=2.08, EC₅₀=17.29 at 6 μM.

TABLE 4

| | | Initial | | Channel |
|---|---|---|---|---|
| | | velocity of | Apparent | activity |
| | | channel | EC₅₀ | increase |
| | | activation | based | for 80 s |
| | | for 4 s (vᵢ) | on | (ΔRFU) |
| | | at 6 μM | vᵢ | at |
| Compound | Structure | (sec⁻¹) | (μM) | 6 μM |
| 107 | 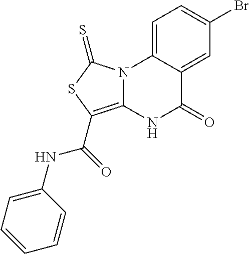 [SMILES: O=C(Nc1ccccc1)c1sc(=S)n2c1[nH]c(=O)c1cc(Br)ccc12] | 5.51 | 12.33 | 3.83 |
| 201 | 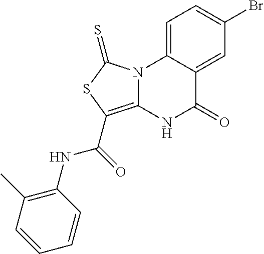 [SMILES: Cc1ccccc1NC(=O)c1sc(=S)n2c1[nH]c(=O)c1cc(Br)ccc12] | 9.40 | 4.60 | 3.68 |
| 202(═TTQC-1) | 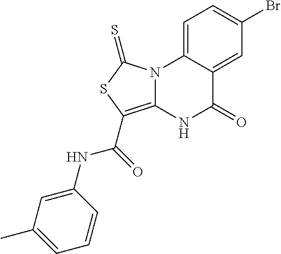 [SMILES: Cc1cccc(NC(=O)c2sc(=S)n3c2[nH]c(=O)c2cc(Br)ccc23)c1] | 9.77 | 5.74 | 4.98 |
| 203 | 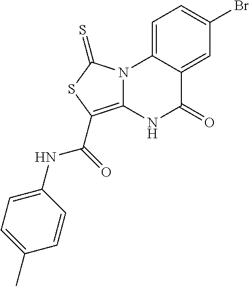 [SMILES: Cc1ccc(NC(=O)c2sc(=S)n3c2[nH]c(=O)c2cc(Br)ccc23)cc1] | 0.71 | NA | 1.41 |
| 204 | 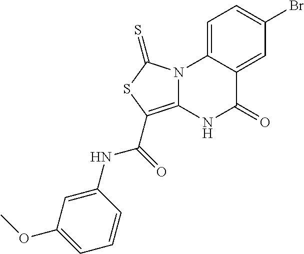 [SMILES: COc1cccc(NC(=O)c2sc(=S)n3c2[nH]c(=O)c2cc(Br)ccc23)c1] | 6.00 | 6.60 | 3.35 |
| 205 | 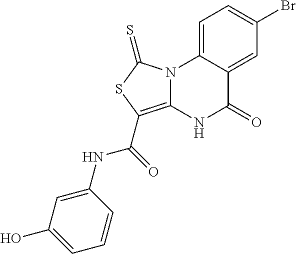 [SMILES: O=C(Nc1cccc(O)c1)c1sc(=S)n2c1[nH]c(=O)c1cc(Br)ccc12] | 4.03 | 11.22 | 4.88 |
| 206 | 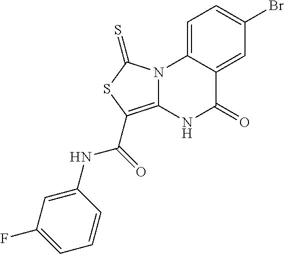 [SMILES: O=C(Nc1cccc(F)c1)c1sc(=S)n2c1[nH]c(=O)c1cc(Br)ccc12] | 12.55 | 1.14 | 2.85 |
| 207 | 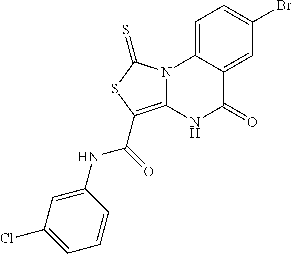 [SMILES: O=C(Nc1cccc(Cl)c1)c1sc(=S)n2c1[nH]c(=O)c1cc(Br)ccc12] | 16.12 | 4.85 | 3.56 |
| 208 | 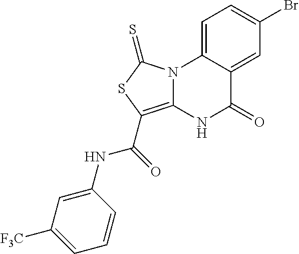 [SMILES: O=C(Nc1cccc(C(F)(F)F)c1)c1sc(=S)n2c1[nH]c(=O)c1cc(Br)ccc12] | 16.77 | 2.89 | 3.35 |
| 209 | 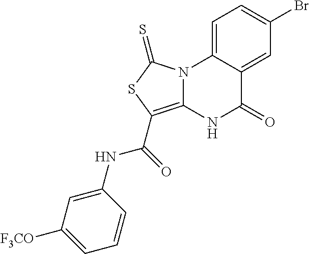 [SMILES: O=C(Nc1cccc(OC(F)(F)F)c1)c1sc(=S)n2c1[nH]c(=O)c1cc(Br)ccc12] | 14.78 | 3.82 | 4.37 |
| 210 | 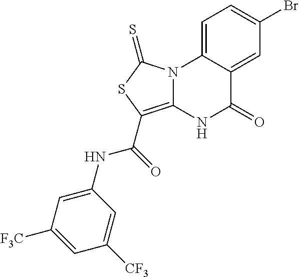 [SMILES: O=C(Nc1cc(C(F)(F)F)cc(C(F)(F)F)c1)c1sc(=S)n2c1[nH]c(=O)c1cc(Br)ccc12] | 2.08 | 17.29 | 2.28 |
| NS11021 | 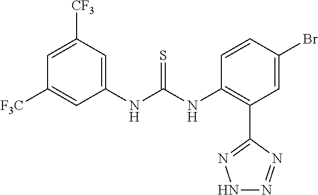 [SMILES: FC(F)(F)c1cc(NC(=S)Nc2ccc(Br)cc2-c2nn[nH]n2)cc(C(F)(F)F)c1] | 1.63 | 14.29 | 1.86 |

### 2.2. Measurement of Macroscopic Current

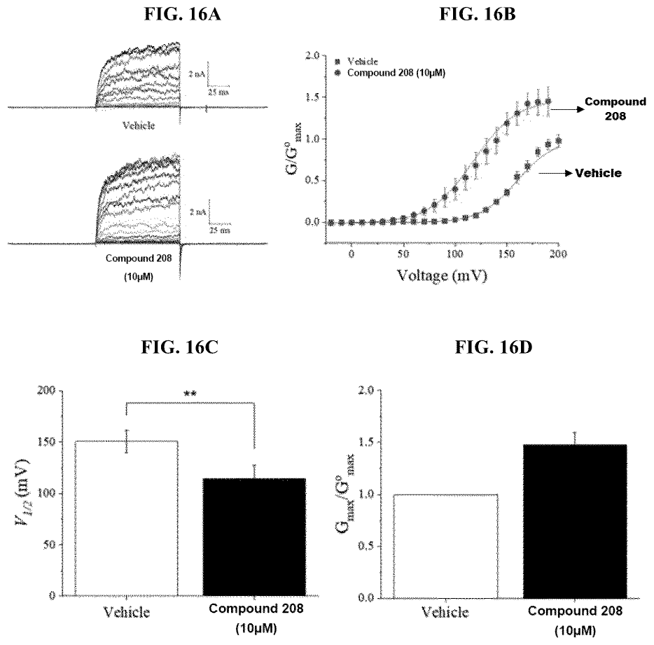

To observe activation of Compound 208 on BK_{Ca}, macroscopic currents were directly measured using patch clamp recordings. Perfusion of Compound 208 shifted the conductance-voltage relationship curve of the BK_{Ca} channels to the left at a single concentration of 10 μM (FIGS. 16A and 16B). The voltage at half activation (V_{1/2}) fitted with the Boltzmann function was shifted to the left by 42.6±7.35 mV, which indicates that the channel was opened more readily upon lower membrane depolarization in the presence of Compound 208 (FIG. 16C). Since the BK_{Ca} channels are closed at the hyperpolarized membrane potential and activated by the depolarized membrane potential, it shows that the channel can be activated with smaller voltage stimulation in the presence of Compound 208. V_{1/2} is relevant to the channel activation Gibbs-free energy (ΔG) according to the Nernst equation: ΔG=−zFV_{1/2}. Negative ΔV_{1/2} shifted to the left indicates a decrease in ΔG, and shows that channel activation is further stabilized by Compound 208 bond (−1.92 kcal/mol). The maximum conductance (Gₘₐₓ) was increased by 1.5±0.11 times compared to the vehicle treatment group, which indicates an increase in the possibility of channel opening or single channel conductance (FIG. 16D).

### 2.3. Effect of Compound 208 on Channel Gating Kinetics

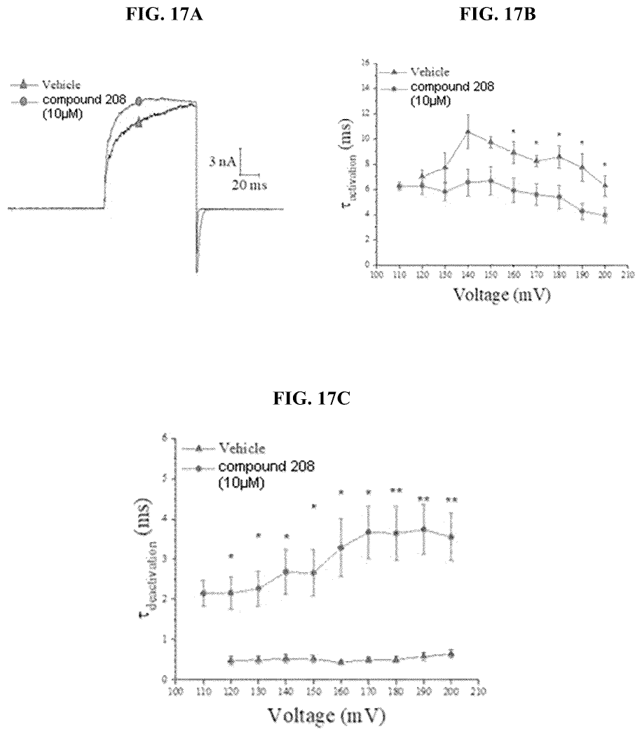

To observe the effect of Compound 208 on channel gating kinetics, the activation (i activation) and deactivation (i deactivation) time constants were induced using exponential equations for the outward current with voltage stimulation and the tail current without voltage stimulation, respectively. Both the τ activation and the τ deactivation were significantly affected by Compound 208. This shows that the channel opening was shortened and the channel closing was delayed due to the Compound 208 bond (see FIGS. 17A to 17C).

### 2.4. Overactive Bladder In Vivo Treatment Effect

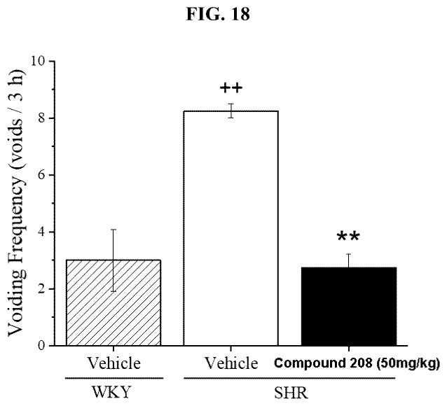

The in vivo treatment effects of the compounds for 12 hours were investigated in spontaneously hypertensive rats (SHR) with OAB symptoms after oral administration. Wistar-Kyoto rats (WKY) were used as the healthy animal control, and urination frequency was increased more than 2.8 times in SHR compared to WKY. Compound 208 reduced the urination frequency in SHR (50 mg/kg) by 33%, which is quite close to the urination frequency of the normal group (see FIG. 18).

1: A composition comprising a compound represented by Formula 1 below, a stereoisomer or a pharmaceutically acceptable salt thereof: 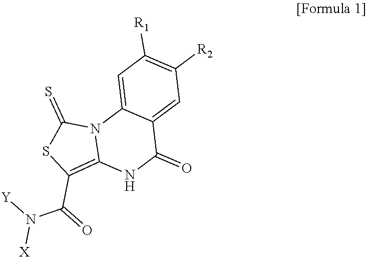
wherein X is methyl, isopropyl, 2-chlorobenzyl, 4-methylbenzyl, 3-methoxybenzyl, 4-methoxybenzyl, cyclopentyl, (tetrahydrofuran-2-yl)methyl, or substituted or unsubstituted phenyl, and Y is hydrogen or piperidine formed by linking with X, and
R₁ and R₂ are each independently hydrogen, methyl, halogen, methoxy or methoxycarbonyl, or R₁ and R₂ are 1,3-dioxolane formed by linking with each other.

2: The composition according to claim 1, wherein the substituted phenyl is 2-methylphenyl, 3-methylphenyl, 4-methylphenyl, 2,4-dimethylphenyl, 5-chloro-2-methylphenyl, 2-ethylphenyl, 4-ethylphenyl, 2-methoxyphenyl, 3-methoxyphenyl, 2,5-dimethoxyphenyl, 2-ethoxyphenyl, 3-ethoxyphenyl, 4-(ethoxycarbonyl)phenyl, 3-fluorophenyl, 2,4-difluorophenyl, 4-bromo-2-fluorophenyl, 2-chlorophenyl, 3-chlorophenyl, 3-(trifluoromethyl)phenyl, 3-(trifluoromethoxy)phenyl or 3,5-bis(trifluoromethyl)phenyl.

3: The composition according to claim 1, wherein R₁ is hydrogen and R₂ is Br.

4: The composition according to claim 1, wherein the compound represented by Formula 1 above is any one selected from the group consisting of the following compounds:
7-bromo-5-oxo-1-thioxo-N-(m-tolyl)-4,5-dihydro-1H-thiazolo[3,4-a]quinazoline-3-carboxamide;
methyl 3-((3-fluorophenyl)carbamoyl)-5-oxo-1-thioxo-4,5-dihydro-1H-thiazolo[3,4-a]quinazoline-8-carboxylate;
ethyl 4-(8-chloro-5-oxo-1-thioxo-4,5-dihydro-1H-thiazolo[3,4-a]quinazoline-3-carboxamido)benzoate;
8-chloro-N-(2-ethylphenyl)-5-oxo-1-thioxo-4,5-dihydro-1H-thiazolo[3,4-a]quinazoline-3-carboxamide;
7-chloro-N-(2-methoxyphenyl)-5-oxo-1-thioxo-4,5-dihydro-1H-thiazolo[3,4-a]quinazoline-3-carboxamide;
7-bromo-N-isopropyl-5-oxo-1-thioxo-4,5-dihydro-1H-thiazolo[3,4-a]quinazoline-3-carboxamide;
N-(5-chloro-2-methylphenyl)-7-methyl-5-oxo-1-thioxo-4,5-dihydro-1H-thiazolo[3,4-a]quinazoline-3-carboxamide;
methyl 3-((2-chlorobenzyl)carbamoyl)-5-oxo-1-thioxo-4,5-dihydro-1H-thiazolo[3,4-a]quinazoline-8-carboxylate;
7-chloro-N-(2,4-dimethylphenyl)-5-oxo-1-thioxo-4,5-dihydro-1H-thiazolo[3,4-a]quinazoline-3-carboxamide;
7-bromo-N-(2,4-dimethylphenyl)-5-oxo-1-thioxo-4,5-dihydro-1H-thiazolo[3,4-a]quinazoline-3-carboxamide;
7-methyl-5-oxo-1-thioxo-N-(o-tolyl)-4,5-dihydro-1H-thiazolo[3,4-a]quinazoline-3-carboxamide;
N-(5-chloro-2-methylphenyl)-5-oxo-1-thioxo-4,5-dihydro-1H-[1,3]dioxolo[4,5-g]thiazolo[3,4-a]quinazoline-3-carboxamide;
methyl 3-((2,4-difluorophenyl)carbamoyl)-5-oxo-1-thioxo-4,5-dihydro-1H-thiazolo[3,4-a]quinazoline-8-carboxylate;
7-bromo-N-(4-methylbenzyl)-5-oxo-1-thioxo-4,5-dihydro-1H-thiazolo[3,4-a]quinazoline-3-carboxamide;
7-bromo-N-(2,5-dimethoxyphenyl)-5-oxo-1-thioxo-4,5-dihydro-1H-thiazolo[3,4-a]quinazoline-3-carboxamide;
7-bromo-5-oxo-N-((tetrahydrofuran-2-yl)methyl)-1-thioxo-4,5-dihydro-1H-[1,3]thiazolo[3,4-a]quinazoline-3-carboxamide;
N-(5-chloro-2-methylphenyl)-7,8-dimethoxy-5-oxo-1-thioxo-4,5-dihydro-1H-thiazolo[3,4-a]quinazoline-3-carboxamide;
N-(2,5-dimethoxyphenyl)-7-methyl-5-oxo-1-thioxo-4,5-dihydro-1H-thiazolo[3,4-a]quinazoline-3-carboxamide;
N-(2-ethylphenyl)-5-oxo-1-thioxo-4,5-dihydro-1H-[1,3]dioxolo[4,5-g]thiazolo[3,4-a]quinazoline-3-carboxamide;
7-bromo-N-(2-chlorobenzyl)-5-oxo-1-thioxo-4,5-dihydro-1H-thiazolo[3,4-a]quinazoline-3-carboxamide;
7-chloro-N-(2-chlorobenzyl)-5-oxo-1-thioxo-4,5-dihydro-1H-thiazolo[3,4-a]quinazoline-3-carboxamide;
7,8-dimethoxy-5-oxo-N-phenyl-1-thioxo-4,5-dihydro-1H-thiazolo[3,4-a]quinazoline-3-carboxamide;
8-chloro-N-cyclopentyl-5-oxo-1-thioxo-4,5-dihydro-1H-thiazolo[3,4-a]quinazoline-3-carboxamide;
N-(2-methoxyphenyl)-5-oxo-1-thioxo-4,5-dihydro-1H-[1,3]dioxolo[4,5-g]thiazolo[3,4-a]quinazoline-3-carboxamide;
8-chloro-N-methyl-5-oxo-1-thioxo-4,5-dihydro-1H-thiazolo[3,4-a]quinazoline-3-carboxamide;
N-(4-bromo-2-fluorophenyl)-8-chloro-5-oxo-1-thioxo-4,5-dihydro-1H-thiazolo[3,4-a]quinazoline-3-carboxamide;
7-chloro-5-oxo-N-((tetrahydrofuran-2-yl)methyl)-1-thioxo-4,5-dihydro-1H-thiazolo[3,4-a]quinazoline-3-carboxamide;
7-chloro-N-(2-ethoxyphenyl)-5-oxo-1-thioxo-4,5-dihydro-1H-thiazolo[3,4-a]quinazoline-3-carboxamide;
8-chloro-N-(3-methoxybenzyl)-5-oxo-1-thioxo-4,5-dihydro-1H-thiazolo[3,4-a]quinazoline-3-carboxamide;
7-bromo-3-(piperidine-1-carbonyl)-1-thioxo-1H-thiazolo[3,4-a]quinazolin-5(4H)-one;
N-(2,4-dimethylphenyl)-7-methyl-5-oxo-1-thioxo-4,5-dihydro-1H-thiazolo[3,4-a]quinazoline-3-carboxamide;
7-bromo-N-(4-ethylphenyl)-5-oxo-1-thioxo-4,5-dihydro-1H-thiazolo[3,4-a]quinazoline-3-carboxamide;
8-chloro-N-(4-methoxybenzyl)-5-oxo-1-thioxo-4,5-dihydro-1H-thiazolo[3,4-a]quinazoline-3-carboxamide;
8-chloro-N-(2,5-dimethoxyphenyl)-5-oxo-1-thioxo-4,5-dihydro-1H-thiazolo[3,4-a]quinazoline-3-carboxamide;
5-oxo-N-phenyl-1-thioxo-4,5-dihydro-1H-thiazolo[3,4-a]quinazoline-3-carboxamide;
8-methyl-5-oxo-N-phenyl-1-thioxo-4,5-dihydro-1H-thiazolo[3,4-a]quinazoline-3-carboxamide;
8-chloro-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-methyl-5-oxo-N-phenyl-1-thioxo-4,5-dihydro-1H-thiazolo[3,4-a]quinazoline-3-carboxamide;
7-chloro-5-oxo-N-phenyl-1-thioxo-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-5-oxo-1-thioxo-N-(p-tolyl)-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

5-8. (canceled)

9: A compound represented by Formula 2 below, a stereoisomer or a pharmaceutically acceptable salt thereof: 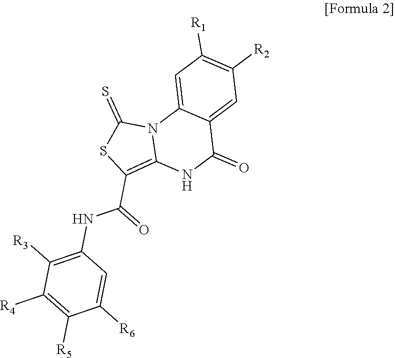
wherein R₁ and R₂ are each independently hydrogen, methyl, or halogen, and R₃, R₄, R₅ and R₆ are each independently hydrogen, methyl, hydroxy, methoxy, halogen, trifluoromethyl or trifluoromethoxy.

10: The compound, or the stereoisomer or the pharmaceutically acceptable salt thereof according to claim 9, wherein R₁ is hydrogen and R₂ is Br.

11: The compound, or the stereoisomer or the pharmaceutically acceptable salt thereof according to claim 9, wherein the compound represented by Formula 2 above is any one compound selected from the group consisting of the following compounds:
8-methyl-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.

12: A method for treating overactive bladder, the method comprising:
administering a composition to a subject in need thereof, the composition comprising:
a compound represented by Formula 1 below, a stereoisomer or a pharmaceutically acceptable salt thereof: 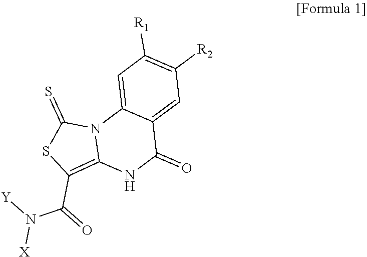
wherein X is methyl, isopropyl, 2-chlorobenzyl, 4-methylbenzyl, 3-methoxybenzyl, 4-methoxybenzyl, cyclopentyl, (tetrahydrofuran-2-yl)methyl, or substituted or unsubstituted phenyl, and Y is hydrogen or piperidine formed by linking with X, and
R₁ and R₂ are each independently hydrogen, methyl, halogen, methoxy or methoxycarbonyl, or R₁ and R₂ are 1,3-dioxolane formed by linking with each other.

13: The method of claim 12, wherein the substituted phenyl is 2-methylphenyl, 3-methylphenyl, 4-methylphenyl, 2,4-dimethylphenyl, 5-chloro-2-methylphenyl, 2-ethylphenyl, 4-ethylphenyl, 2-methoxyphenyl, 3-methoxyphenyl, 2,5-dimethoxyphenyl, 2-ethoxyphenyl, 3-ethoxyphenyl, 4-(ethoxycarbonyl)phenyl, 3-fluorophenyl, 2,4-difluorophenyl, 4-bromo-2-fluorophenyl, 2-chlorophenyl, 3-chlorophenyl, 3-(trifluoromethyl)phenyl, 3-(trifluoromethoxy)phenyl or 3,5-bis(trifluoromethyl)phenyl.

14: The method of claim 12, wherein R₁ is hydrogen and R₂ is Br.

15: The method of claim 12, wherein the compound represented by Formula 1 above is any one selected from the group consisting of the following compounds:
7-bromo-5-oxo-1-thioxo-N-(m-tolyl)-4,5-dihydro-1H-thiazolo[3,4-a]quinazoline-3-carboxamide;
methyl 3-((3-fluorophenyl)carbamoyl)-5-oxo-1-thioxo-4,5-dihydro-1H-thiazolo[3,4-a]quinazoline-8-carboxylate;
ethyl 4-(8-chloro-5-oxo-1-thioxo-4,5-dihydro-1H-thiazolo[3,4-a]quinazoline-3-carboxamido)benzoate;
8-chloro-N-(2-ethylphenyl)-5-oxo-1-thioxo-4,5-dihydro-1H-thiazolo[3,4-a]quinazoline-3-carboxamide;
7-chloro-N-(2-methoxyphenyl)-5-oxo-1-thioxo-4,5-dihydro-1H-thiazolo[3,4-a]quinazoline-3-carboxamide;
7-bromo-N-isopropyl-5-oxo-1-thioxo-4,5-dihydro-1H-thiazolo[3,4-a]quinazoline-3-carboxamide;
N-(5-chloro-2-methylphenyl)-7-methyl-5-oxo-1-thioxo-4,5-dihydro-1H-thiazolo[3,4-a]quinazoline-3-carboxamide;
methyl 3-((2-chlorobenzyl)carbamoyl)-5-oxo-1-thioxo-4,5-dihydro-1H-thiazolo[3,4-a]quinazoline-8-carboxylate;
7-chloro-N-(2,4-dimethylphenyl)-5-oxo-1-thioxo-4,5-dihydro-1H-thiazolo[3,4-a]quinazoline-3-carboxamide;
7-bromo-N-(2,4-dimethylphenyl)-5-oxo-1-thioxo-4,5-dihydro-1H-thiazolo[3,4-a]quinazoline-3-carboxamide;
7-methyl-5-oxo-1-thioxo-N-(o-tolyl)-4,5-dihydro-1H-thiazolo[3,4-a]quinazoline-3-carboxamide;
N-(5-chloro-2-methylphenyl)-5-oxo-1-thioxo-4,5-dihydro-1H-[1,3]dioxolo[4,5-g]thiazolo[3,4-a]quinazoline-3-carboxamide;
methyl 3-((2,4-difluorophenyl)carbamoyl)-5-oxo-1-thioxo-4,5-dihydro-1H-thiazolo[3,4-a]quinazoline-8-carboxylate;
7-bromo-N-(4-methylbenzyl)-5-oxo-1-thioxo-4,5-dihydro-1H-thiazolo[3,4-a]quinazoline-3-carboxamide;
7-bromo-N-(2,5-dimethoxyphenyl)-5-oxo-1-thioxo-4,5-dihydro-1H-thiazolo[3,4-a]quinazoline-3-carboxamide;
7-bromo-5-oxo-N-((tetrahydrofuran-2-yl)methyl)-1-thioxo-4,5-dihydro-1H-[1,3]thiazolo[3,4-a]quinazoline-3-carboxamide;
N-(5-chloro-2-methylphenyl)-7,8-dimethoxy-5-oxo-1-thioxo-4,5-dihydro-1H-thiazolo[3,4-a]quinazoline-3-carboxamide;
N-(2,5-dimethoxyphenyl)-7-methyl-5-oxo-1-thioxo-4,5-dihydro-1H-thiazolo[3,4-a]quinazoline-3-carboxamide;
N-(2-ethylphenyl)-5-oxo-1-thioxo-4,5-dihydro-1H-[1,3]dioxolo[4,5-g]thiazolo[3,4-a]quinazoline-3-carboxamide;
7-bromo-N-(2-chlorobenzyl)-5-oxo-1-thioxo-4,5-dihydro-1H-thiazolo[3,4-a]quinazoline-3-carboxamide;
7-chloro-N-(2-chlorobenzyl)-5-oxo-1-thioxo-4,5-dihydro-1H-thiazolo[3,4-a]quinazoline-3-carboxamide;
7,8-dimethoxy-5-oxo-N-phenyl-1-thioxo-4,5-dihydro-1H-thiazolo[3,4-a]quinazoline-3-carboxamide;
8-chloro-N-cyclopentyl-5-oxo-1-thioxo-4,5-dihydro-1H-thiazolo[3,4-a]quinazoline-3-carboxamide;
N-(2-methoxyphenyl)-5-oxo-1-thioxo-4,5-dihydro-1H-[1,3]dioxolo[4,5-g]thiazolo[3,4-a]quinazoline-3-carboxamide;
8-chloro-N-methyl-5-oxo-1-thioxo-4,5-dihydro-1H-thiazolo[3,4-a]quinazoline-3-carboxamide;
N-(4-bromo-2-fluorophenyl)-8-chloro-5-oxo-1-thioxo-4,5-dihydro-1H-thiazolo[3,4-a]quinazoline-3-carboxamide;
7-chloro-5-oxo-N-((tetrahydrofuran-2-yl)methyl)-1-thioxo-4,5-dihydro-1H-thiazolo[3,4-a]quinazoline-3-carboxamide;
7-chloro-N-(2-ethoxyphenyl)-5-oxo-1-thioxo-4,5-dihydro-1H-thiazolo[3,4-a]quinazoline-3-carboxamide;
8-chloro-N-(3-methoxybenzyl)-5-oxo-1-thioxo-4,5-dihydro-1H-thiazolo[3,4-a]quinazoline-3-carboxamide;
7-bromo-3-(piperidine-1-carbonyl)-1-thioxo-1H-thiazolo[3,4-a]quinazolin-5(4H)-one;
N-(2,4-dimethylphenyl)-7-methyl-5-oxo-1-thioxo-4,5-dihydro-1H-thiazolo[3,4-a]quinazoline-3-carboxamide;
7-bromo-N-(4-ethylphenyl)-5-oxo-1-thioxo-4,5-dihydro-1H-thiazolo[3,4-a]quinazoline-3-carboxamide;
8-chloro-N-(4-methoxybenzyl)-5-oxo-1-thioxo-4,5-dihydro-1H-thiazolo[3,4-a]quinazoline-3-carboxamide;
8-chloro-N-(2,5-dimethoxyphenyl)-5-oxo-1-thioxo-4,5-dihydro-1H-thiazolo[3,4-a]quinazoline-3-carboxamide;
5-oxo-N-phenyl-1-thioxo-4,5-dihydro-1H-thiazolo[3,4-a]quinazoline-3-carboxamide;
8-methyl-5-oxo-N-phenyl-1-thioxo-4,5-dihydro-1H-thiazolo[3,4-a]quinazoline-3-carboxamide;
8-chloro-5-oxo-N-phenyl-1-thioxo-4,5-dihydro-1H-thiazolo[3,4-a]quinazoline-3-carboxamide;
8-bromo-5-oxo-N-phenyl-1-thioxo-4,5-dihydro-1H-thiazolo[3,4-a]quinazoline-3-carboxamide;
7-methyl-5-oxo-N-phenyl-1-thioxo-4,5-dihydro-1H-thiazolo[3,4-a]quinazoline-3-carboxamide;
7-chloro-5-oxo-N-phenyl-1-thioxo-4,5-dihydro-1H-thiazolo[3,4-a]quinazoline-3-carboxamide;
7-bromo-5-oxo-N-phenyl-1-thioxo-4,5-dihydro-1H-thiazolo[3,4-a]quinazoline-3-carboxamide;
7-bromo-5-oxo-1-thioxo-N-(o-tolyl)-4,5-dihydro-1H-thiazolo[3,4-a]quinazoline-3-carboxamide;
7-bromo-5-oxo-1-thioxo-N-(p-tolyl)-4,5-dihydro-1H-thiazolo[3,4-a]quinazoline-3-carboxamide;
7-bromo-N-(3-methoxyphenyl)-5-oxo-1-thioxo-4,5-dihydro-1H-thiazolo[3,4-a]quinazoline-3-carboxamide;
7-bromo-N-(3-hydroxyphenyl)-5-oxo-1-thioxo-4,5-dihydro-1H-thiazolo[3,4-a]quinazoline-3-carboxamide;
7-bromo-N-(3-fluorophenyl)-5-oxo-1-thioxo-4,5-dihydro-1H-thiazolo[3,4-a]quinazoline-3-carboxamide;
7-bromo-N-(3-chlorophenyl)-5-oxo-1-thioxo-4,5-dihydro-1H-thiazolo[3,4-a]quinazoline-3-carboxamide;
7-bromo-5-oxo-1-thioxo-N-(3-(trifluoromethyl)phenyl)-4,5-dihydro-1H-thiazolo[3,4-a]quinazoline-3-carboxamide;
7-bromo-5-oxo-1-thioxo-N-(3-(trifluoromethoxy)phenyl)-4,5-dihydro-1H-thiazolo[3,4-a]quinazoline-3-carboxamide; and
N-(3,5-bis(trifluoromethyl)phenyl)-7-bromo-5-oxo-1-thioxo-4,5-dihydro-1H-thiazolo[3,4-a]quinazoline-3-carboxamide.
